(19) United States
(12) Patent Application Publication
(10) Pub. No.: US 2003/0190600 A1
(43) Pub. Date: Oct. 9, 2003

(54) Carcinoembryonic antigen cell adhesion molecule 1 (CEACAM1) structure and uses thereof in drug identification and screening

(75) Inventors: Kathryn V. Holmes, Golden, CO (US); Bruce D. Zelus, Lakewood, CO (US); Kemin Tan, Waltham, MA (US); Jia-Huai Wang, Belmont, MA (US); Rob Meijers, Somerville, MA (US)

Correspondence Address: Sheridan Ross P. C., 1560 Broadway, Suite 1200, Denver, CO, 80202-5141, US

(73) Assignees: Dana-Faber Cancer Institute; The Regents of the University of Colorado

(21) Appl. No.: 10/118,471

(22) Filed: Apr. 5, 2002

(51) Int. Cl. A01N 37/18; A61K 38/00; C12Q 1/70; G01N 33/53; C07K 1/00; C07K 14/00; C07K 17/00

(52) U.S. Cl. 435/5; 435/7.1; 530/350; 514/2

(57) ABSTRACT

Disclosed are novel crystal structures of a carcinoembryonic cell adhesion antigen functional domain that is characterized as having a unique N-terminal domain structure, called a CC′ loop. This tertiary structure is used in a number of screening methods for identifying candidate molecules that have a binding affinity for the tertiary structure of the CC′ loop. Pharmaceutical preparations that include one or more of such identified candidate may then be provided and used in treatments for bacterial infections, dysentery, angiogenesis, immune cell mediated disease, and related conditions thereto.

The United States Government may own rights to the invention as research relevant to its development was funded by NIH Grants GM56008, HL48675, AI25231, and HL54734.

### BACKGROUND OF THE INVENTION

CEACAM1 is a member of the carcinoembryonic antigen (CEA) family. Isoforms of murine CEACAM1 serve as receptors for mouse hepatitis virus (MHV), a murine coronavirus.

Carcinoembryonic antigen (CEA; CD66e) was initially discovered as a tumor antigen (Gold and Freedman, 1965). A large group of related glycoproteins is now called the CEA family within Ig superfamily (IgSF). These anchored or secreted glycoproteins are expressed by epithelial cells, leukocytes, endothelial cells and placenta (Hammarstrom, 1999). In humans, the CEA family contains 29 genes or pseudogenes. The revised nomenclature of this family of glycoproteins was recently summarized (Beauchemin et al., 1999). The CEA family consists of the CEACAM (CEA-related cell adhesion molecule) and PSG (pregnancy-specific glycoprotein) subfamilies whose proteins share many common structural features (Hammarstrom, 1999).

CEACAM1 (CD66a) is the most highly conserved member of the CEA family. Most species have only one CEACAM1 gene, but mice have two closely related genes called CEACAM1 and CEACAM2 (Beauchemin et al., 1999). CEACAM1 has many important biological functions. It is a potent vascular endothelial growth factor (Ergun et al., 2000) and a growth inhibitor in tumor cells (Izzi et al., 1999); plays a key role in differentiation of mammary glands (Huang et al., 1999); is an early marker of T cell activation; and modulates the functions of murine T lymphocytes (Morales et al., 1999; Nakajima et al., 2002). Human CEACAM1 is one of several human CEACAM proteins that serve as receptors for virulent strains of Neisseria gonorrhoeae, Neisseria meningitidis, and Hemophilus influenzae (Bos et al., 1999; Virji et al., 2000; Virji et al., 1999).

In mice four isoforms of CEACAM1 generated by alternative mRNA splicing have either 2 [D1,D4] or 4 [D1-D4] Ig-like domains on cell surface, a transmembrane segment and either a short or a long cytoplasmic tail (Beauchemin et al., 1999). The long tail contains a modified ITIM (immunoreceptor tyrosine based inhibition motif)-like motif. Tyrosine phosphorylation of this motif is associated with signaling (Huber et al., 1999), but the natural ligands for the ecto-domain and the modulation of gene expression by CEACAM1 signaling are not well understood.

All four isoforms of murine CEACAM1a as well as murine CEACAM2 can serve as receptors for mouse hepatitis virus (MHV) strain A59 (MHV-A59) when the recombinant murine proteins are expressed at high levels in a hamster cell line (BHK) (Dveksler et al., 1993a; Dveksler et al., 1991; Nedellec et al., 1994). MHVs are large, enveloped, positive-stranded RNA viruses in the Coronaviridae family in the order Nidovirales. Various MHV strains cause diarrhea, hepatitis, respiratory, neurological and immunological disorders in mice. Infection is initiated by binding of the 180 kDa spike glycoprotein (S) on the viral envelope to a CEACAM glycoprotein on a murine cell membrane. Most inbred mouse strains are highly susceptible to MHV infection, but SJL/J mice are highly resistant. Susceptible strains are homozygous for the CEACAM1a allele that encodes the principal MHV receptor, while SJL/J mice are homozygous for the CEACAM1b allele. CEACAM1b proteins have weaker MHV binding and receptor activities than CEACAM1a proteins (Ohtsuka et al., 1996; Rao et al., 1997; Wessner et al., 1998). Humans have only one CEACAM1 allele.

What is known about the family of CEACAM1a proteins is that MHV strains utilize the murine CEACAM1a proteins as receptors (Compton, S. R. (1994), Virology, 203:197-201; Dueksler et al. (1993) J. Virol, 67:1-8). The spike (S) glycoprotein of MHV attaches to the N domain (D1) of CEACAM1a (Dveksler, et al., 1993, PNAS 90:1716-20). Mutational analysis showed that the virus MHV, binds to the B—C—C— region of domain 1 of the CEACAM1a protein (Rao, et al. (1997), Virology, 229:336-48; Wessner, et al. (1998), J. Virol. 72:194-48). However, extensive N-linked glycosylation has hampered crystallization of any CEA proteins for structural analysis. A need to continues to exist in the arts for the location of the structure of this important family of proteins, as to do so would permit the development of a broad spectrum of therapeutic agents for viral, bacterial and carcinogenic pathologies.

### SUMMARY OF THE INVENTION

The present invention, in a general and overall sense, relates to the identification of a uniquely crystalline structure of a biologically important molecule that to this time had been precluded by the extensive glycosylation inherent in the native CEA antigen. The structure of the biologically active CC′ loop of the N-terminal domain (domain 1) could not have been predicted based on a comparison of its linear amino acid sequence with that of any other known structure of any other protein in the database. The identification of this structure may be used in the selection and screening of agents for use in treatment of viral, bacterial, immunological diseases, malignancies and abnormal blood vessel growth. The crystal structure of soluble murine sCEACAM1a[1,4], is composed of two Ig-like domains. This protein has virus neutralizing activity. Its N-terminal domain has a uniquely folded CC′ loop that encompasses key virus-binding residues, these are KGNTTAIDKE. This is the first atomic structure of any member of the CEA family, and provides a prototypic architecture for functional identification of all other CEA family members. The structural basis of virus receptor activities of murine CEACAM1 proteins, binding of Neisseria to human CEACAM1, and other homophilic and heterophilic interactions of CEA family members is disclosed in the present invention.

The crystal structure is of the soluble ecto-domain of an isoform of murine CEACAM1a that consists of domains 1 and 4, (designated msCEACAM1a[1,4] hereafter) and has MHV neutralizing activity. The relationship of the structure of the msCEACAM1a[1,4] glycoprotein to its MHV binding and neutralizing activities is examined and described here. Based on the structure of msCEACAM1a[1,4], the structures of human CEA as well as other CEA family members is provided, and the biological use of these features disclosed.

The term “fragment”, as applied herein to a peptide, refers to at least 7 contiguous amino acids, preferably about 14 to 16 contiguous amino acids, or up to more than 40 contiguous amino acids in length. Such peptides can be produced by well-known methods to those skilled in the art, such as, for example, by proteolytic cleavage, genetic engineering or chemical synthesis.

Unless defined otherwise, the scientific and technological terms and nomenclature used herein have the same meaning as commonly understood by a person of ordinary skill to which the invention pertains. Generally, the procedures for cell cultures, infection, molecular biology methods and the like are common methods used in the art. Such standard techniques can be found in reference manuals such as for example Sambrook et al. (1989, Molecular Cloning—A Laboratory Manual, Cold Spring Harbor Laboratories) and Ausubel et al. (1994. Current protocols in Molecular Biology, Wiley, N.Y.).

As used herein, “nucleic acid molecule”, refers to a polymer of nucleotides. Non-limiting examples thereof include DNA (e.g. genomic DNA, cDNA), RNA molecules (e.g. mRNA) and chimeras thereof. The nucleic acid molecule can be obtained by cloning techniques or synthesized. DNA can be double-stranded or single-stranded (coding strand or non-coding strand [antisense]). RNA can be single-stranded or double-stranded, or partially double stranded.

The nucleic acid (e.g. DNA or RNA) for practicing the present invention may be obtained according to well known methods.

The term “DNA segment” is used herein to refer to DNA molecule comprising a linear stretch or sequence of nucleotides. This sequence when read in accordance with the genetic code, can encode a linear stretch or sequence of amino acids which can be referred to as a polypeptide, protein, protein fragment and the like.

As used herein, “oligonucleotides” or “oligos” define a molecule having two or more nucleotides (ribo or deoxyribonucleotides). The size of the oligo will be dictated by the particular situation and ultimately on the particular use thereof and adapted accordingly by the person of f ordinary skill. An oligonucleotide can be synthetised chemically or derived by cloning according to well known methods.

The nucleic acid (e.g. DNA or RNA) for practicing the present inventions may be obtained according to well known methods.

The term “DNA” molecule or sequence refers to a molecule generally comprised of the deoxyribonucleotides adenine (A), guanine (G), thymine (T), and/or cytosine (C), which in a double-stranded form, can comprise or include a “regulatory element” according to the present invention, as the term is defined herein. “DNA” can be found in linear DNA molecules or fragments, viruses, plasmids, vectors, chromosomes or synthetically derived DNA. As used herein, particular double-stranded DNA sequences may be described according to the normal convention of giving only the sequence in the 5′ to 3′ direction. The same applies to single stranded DNA sequences. As well known in the art, DNA can also be found as circular molecules.

“Nucleic acid hybridization” refers generally to the hybridization of two single stranded nucleic acid molecules having complementary base sequences, which under appropriate conditions will form a thermodynamically favored double-stranded structure. Examples of hybridization conditions can be found in the two laboratory manuals referred above (Sambrook et al., 1989, supra and Ausubel et al., 1989, supra) and are commonly known in the art. In the case of a hybridization to a nitrocellulose filter, as for example in the well known Southern blotting procedure, a nitrocellulose filter can be incubated overnight at 65° C. with a labelled probe in a solution containing 50% formamide, high salt (5 x SSC or 5×SSPE), 5× Denhardt's solution, 1% SDS, and 100 μg/ml denatured carrrier DNA (e.g. salmon sperm DNA). The non-specifically binding probe can then be washed off the filter by several washes in 0.2×SSC/0.1% SDS at a temperature which is selected in view of the desired stringency: room temperature (low stringency), 42° C. (moderate stringency) or 65° C. (high stringency). The selected temperature is based on the melting temperature (Tm) of the DNA hybrid. Of course, RNA-DNA hybrids can also be formed and detected. In such cases, the conditions of hybridization and washing can be adapted according to well known methods by the person of ordinary skill. Stringent conditions will be preferably used (Sambrook et al., 1989, sutpra).

Probes of the invention can be utilized with naturally occurring sugar-phosphate backbones as well as modified backbones including phosphorothioates, dithionates, alkyl phosphonates and ∀-nucleotides and the like. Modified sugar-phosphate backbones are generally taught by Miller, 1988, Ann. Reports Med. Chem. 23:295 and Moran et al., 1987, Nucleic acid molecule. Acids Res., 14:5019. Probes of the invention can be constructed of either ribonucleic acid (RNA) or deoxyribonucleic acid (DNA).

The types of detection methods in which probes can be used include Southern blots (DNA detection), dot or slot blots (DNA, RNA), and Northern blots (RNA detection). Although less preferred, labelled proteins could also be used to detect a particular nucleic acid sequence to which it binds. Other detection methods include kits containing probes on a dipstick setup and the like.

Probes can be labelled according to numerous well known methods (Sambrook et al., 1989, supra). Non-limiting examples of labels include ³H, ¹⁴C, ³²P, and ³⁵S. Non-limiting examples of detectable markers include ligands, fluorophores, chemiluminescent agents, enzymes, and antibodies. Other detectable markers for use with probes, which can enable an increase in sensitivity of the method of the invention, include biotin and radionucleotides. It will become evident to the person of ordinary skill that the choice of a particular label dictates the manner in which it is bound to the probe.

As commonly known, radioactive nucleotides can be incorporated into probes of the invention by several methods. Non-limiting examples thereof include kinasing the 5′ ends of the probes using gamma ³²P ATP and polynucleotide kinase, using the Klenow fragement of Pol 1 of E. coli in the presence of radioactive dNTP (e.g. uniformly labelled DNA probe using random oligonucleotide primers in low-melt gels), using the SP6/T7 system to transcribe a DNA segment in the presence of one or more radioactive NTP, and the like.

As used herein, a “primer” defines an oligonucleotide which is capable of annealing to a target sequence, thereby creating a double stranded region which can serve as an initiation point for DNA synthesis under suitable conditions. In a particularly preferred embodiment, the primer is a single stranded DNA molecule.

Amplification of a selected, or target, nucleic acid sequence may be carried out by a number of suitable methods. See generally Kwoh et al., 1990, Am. Biotechnol. Lab 8:14-25. Numerous amplification techniques have been described and can be readily adapted to suit particular needs of a person of ordinary skill. Non-limiting examples of amplification techniques include polymerase chain reaction (PCR), ligase chain reaction (LCR), strand displacement amplification (SDA), transcription-based amplification, the Qβ replicase system and NASBA (Kwoh et al., 1989, Proc. Natl. Acad. Sci. USA 86, 1173-1177; Lizardi et al., 1988, BioTechnology 6:1197-1202; Malek et al., 1994, Methods Mol. Biol., 28:253-260; and Sambrook et al., 1989, supra). Preferably, amplification will be carried out using PCR.

Polymerase chain reaction (PCR) is carried out in accordance with known techniques. See, e.g., U.S. Pat. Nos. 4,683,195; 4,683,202; 4,800,159; and 4,965,188 (the disclosures of all three U.S. patents are incorporated herein by reference). In general, PCR involves, a treatment of a nucleic acid sample (e.g., in the presence of a heat stable DNA polymerase) under hybridizing conditions, with one oligonucleotide primer for each strand of the specific sequence to be detected. An extension product of each primer for each strand of the specific sequence to be detected. An extension product of each primer which is synthesized is complementary to each of the two nucleic acid strands, with the primers sufficiently complementary to each strand of the specific sequence to hybridize therewith. The extension product synthesized from each primer can also serve as a template for further synthesis of extension products using the same primers. Following a sufficient number of rounds of synthesis of extension products, the sample is analysed to assess whether the sequence or sequences to be detected are present. Detection of the amplified sequence may be carried out by visualization following EtBr staining of the DNA following gel electrophores, or using a detectable label in accordance with known techniques, and the like. For a review on PCR techniques (see PCR Protocols, A Guide to Methods and Amplifications, Micheal et al. Eds, Acad. Press, 1999).

Ligase chain reaction (LCR) is carried out in accordance with known techniques (Weiss, 1991, Science 254:1292). Adaptation of the protocol to meet the desired needs can be carried out by a person of ordinary skill. Strand displacement amplification (SDA) is also carried out in accordance with known techniques or adaptations thereof to meet the particular needs (Walker et al., 1992, Proc. Natl. Acad. Sci. USA 89:392-396; and ibid., 1992, Nucleic Acids Res. 20:1691-1696).

As used herein, the term “gene” is well known in the art and relates to a nucleic acid sequence defining a single protein or polypeptide. A “structural gene” defines a DNA sequence which is transcribed into RNA and translated into a protein having a specific amino acid sequence thereby giving rise to a specific polypeptide or protein. It will be readily recognized by the person of ordinary skill, that the nucleic acid sequence of the present invention can be incorporated into any one of numerous established kit formats which are well known in the art.

A “heterologous” (e.g. a heterologous gene) region of a DNA molecule is a subsegment of DNA within a larger segment that is not found in association therewith in nature. The term “heterologous” can be similarly used to define two polypeptide segments not joined together in nature. Non-limiting examples of heterologous genes include reporter genes such as luciferase, chloramphenicol acetyl transferase, beta-galactosidase, and the like which can be juxtaposed or joined to heterologous control regions or to heterologous polypeptides.

The term “vector” is commonly known in the art and defines a plasmid DNA, phage DNA, viral DNA and the like, which can serve as a DNA vehicle into which DNA of the present invention can be cloned. Numerous types of vectors exist and are well known in the art.

The term “expression” defines the process by which a gene is transcribed into one or more mRNAs (transcription), the mRNA is then being translated (translation) into one polypeptide (or protein) or more.

The terminology “expression vector” defines a vector or vehicle as described above but designed to enable the expression of an inserted sequence following transformation into a host. The cloned gene (inserted sequence) is usually placed under the control of control element sequences such as promoter sequences. The placing of a cloned gene under such control sequences is often referred to as being operably linked to control elements or sequences.

Operably linked sequences may also include two segments that are transcribed onto the same RNA transcript. Thus, two sequences, such as a promoter and a “reporter sequence” are operably linked if transcription commencing in the promoter will produce an RNA transcript of the reporter sequence. In order to be “operably linked” it is not necessary that two sequences be immediately adjacent to one another.

Expression control sequences will vary depending on whether the vector is designed to express the operably linked gene in a prokaryotic or eukaryotic host or both (shuttle vectors) and can additionally contain transcriptional elements such as enhancer elements, termination sequences, tissue-specificity elements, and/or translational initiation and termination sites.

Prokaryotic expression systems are useful for the preparation of large quantities of the protein encoded by the DNA sequence of interest. This protein can be purified according to standard protocols that take advantage of the intrinsic properties thereof, such as size and charge (e.g. SDS gel electrophoresis, gel filtration, centrifugation, ion exchange chromatography, reverse phase chromatography, etc.). In addition, the protein of interest can be purified via affinity chromatography using polyclonal or monoclonal antibodies or nickel affinity chromatography.

The DNA construct can be a vector comprising a promoter that is operably linked to an oligonucleotide sequence, which is in turn, operably linked to a heterologous gene, such as the gene for the luciferase reporter molecule. “Promoter” refers to a DNA regulatory region capable of binding directly or indirectly to RNA polymerase in a cell and and initiating transcription of a downstream (3′ direction) coding sequence. For purposes of the present invention, the promoter is bound at its 3′ terminus by the transcription initiation site and extends upstream (5′ direction) to include the minimum number of bases or elements necessary to initiate transcription at levels detectable above background. Within the promoter will be found a transcription initiation site (conveniently defined by mapping with S1 nuclease), as well as protein binding domains (cosensus sequences) responsible for the binding of RNA polymerase. Eukaryotic promoters will often, but not always, contain “TATA” boxes and “CCAT” boxes. Prokaryotic promoters contain −10 and −35 consensus sequences, which serve to initiate transcription and the transcript products contain Shine-Dalgarno sequences, which serve as ribosome binding references during translation initiation.

As used herein, the designation “functional derivitave”, in the context of a functional derivative denotes, in the context of a functional derivative of a sequence whether a nucleic acid or amino acid sequence, a molecule that retains a biological activity (either function or structural) that is substantially similar to that of the original sequence (e.g. acting as receptor for viral infection). This functional derivative or equivalent may be a natural derivative or may be prepared synthetically. Such derivatives include amino acid sequences having substitutions, deletions, or additions of one or more amino acids, provided that the biological activity of the protein is conserved. The same applies to derivatives of nucleic acid sequences which can have substitutions, deletions, or additions of one or more nucleotides, provided that the biological activity of the sequence is generally maintained. When relating to a protein sequence, the substituting amino acid has chemico-physical properties which are similar to those of the substituted amino acid. The similar chemico-physical properties include similarities in charge, bulkiness, hydrophobicity, hydrophilicity and the like. The term “functional derivatives” is intended to include “fragments”, “segments”, “variants”, “analogs”, or “chemical derivatives” of the subject matter of the present invention.

As well-known in the art, a conservative mutation or substitution of an amino acid refers to mutation or substitution which maintains: 1) the structure of the backbone of the polypeptide (e.g. a beta sheet or alpha-helical structure); 2) the charge or hydrophobicity of the amino acid; or 3) the bulkiness of the side chain. More specifically, the well-known terminologies “hydrophilic residues” relate to serine or threonine. “Hydrophobic residues” refer to leucine, isoleucine, lysine, arginine or histidine. Negatively charged residues” refer to aspartic acid or glutamic acid. Residues having “bulky side chains” refer to phenylalanine, tryptophan or tyrosine.

The term “variant” refers herein to a protein or nucleic acid molecule which is substantially similar in structure and biological activity to the protein, peptide, or nucleic acid described the present invention.

The term “allele” defines an alternative form of a gene that occupies a given locus on a chromosome. Non-limiting examples thereof are exemplified with murine CEACAM1^{a} and CEACAM1^{b}.

As commonly known, a “mutation” is a detectable change in the genetic material which can be transmitted to a daughter cell. As well known, a mutation can be, for example, a detectable change in one or more deoxyribonucleotide or amino acid. For example, nucleotides or amino acids can be added, deleted, substituted for, inverted, or transposed to a new position. Spontaneous mutations and experimentally induced mutations exist. The result of a mutations of nucleic acid or amino acid molecule is a mutant molecule. A mutant polypeptide can be encoded from this mutant nucleic acid molecule.

It shall be understood that the “in vivo” experimental model can also be used to carry out an “in vitro” assay. For example, cellular extracts from the transgenic mice of the present invention can be prepared and used in one of the in vitro method of the present invention or an in vitro method known in the art. Such assay could be used to compare the infectious potential of infectious agents on extracts prepared from knock-out versus wild type CEACAM1 mice.

As used herein in the recitation “indicator cells” refers to cells that express, in one particular embodiment, the CEACAM1 glycoprotein or domains thereof which interact with a viral protein or other cellular protein which is directly or indirectly involved in infection by the virus or other molecular interactions of CEACAM1, and wherein an interaction between these proteins or interacting domains thereof is coupled to an identifiable or selectable phenotype or characteristic such that it provides an assessment of the interaction between same. Such indicator cells can be used in the screening assays of the present invention. In certain embodiments, the indicator cells have been engineered so as to express a chosen derivative, fragment, homologue, or mutant of these interacting domains. The cells can be yeast cells or preferably higher eukaryotic cells such as mammalian cells (WO 96/41169).

A host cell or indicator cell has been “transfected” by exogenous or heterologous DNA (e.g. a DNA construct) when such DNA has been introduced inside the cell. The transfecting DNA may or may not be integrated (covalently linked) into chromosomal DNA making up the genome of the cell. In prokaryotes, yeast, and mammalian cells for example, the transfecting DNA may be maintained on an episomal cell element, such as a plasmid. With respect to eukaryotic cells, a stably transfected cell is one in which the transfecting DNA has become integrated into a chromosome so that it is inherited by daughter cells through chromosome replication. This stability is demonstrated by the ability of the eukaryotic cell to establish cell lines or clones comprised of a population of daughter cells containing the transfecting DNA. Transfection methods are well known in the art (Sambrook et al., 1989, supra; Ausubel et al., 1994 supra).

C C′ loop of human CEACAM1 (10a.a)D1

SEQ ID NO: 1

K-G-E-R-V-D-G-N-R-Q
1 10
D1 loop, human CEACAM1 (1-107 aa)

SEQ ID NO: 2

Q-L-T-T-E-S-M-P-F-N-V-A-E-G-K-E-V-L-L-L-V-H-N-L-P
Q-Q-L-F-G-Y-S-W-V-K-G-E-R-V-D-G-N-R-Q-I-V-G-Y-A-I
G-T-Q-Q-A-T-P-G-P-A-N-S-G-R-E-T-I-Y-P-N-A-S-L-L-I
Q-N-V-T-Q-N-D-T-G-F-Y-T-L-Q-V-I-K-S-D-L-V-N-E-E-A
T-G-O-Q-F-H-V-Y

### BRIEF DESCRIPTION OF THE FIGURES

FIG. 1. Stereo view of the ribbon drawing of msCEACAM1a [1,4] which contains two Ig-like domains. The CC′-loop in the N-terminal domain (D1) which is involved in binding of MHV and other ligands is highlighted in yellow. The predicted key virus-binding residue Ile41 on the CC′ loop is shown in ball-and-stick style. The FG loop of D1, another biologically important element is also shown. The carbohydrate moieties are drawn in ball-and-stick style. The glycan at Asn70 that is conserved in the whole CEA family is labeled. The figure was prepared using MOLSCRIPT®(Krulis, 1991).

FIG. 2(A)-2(C). Superposition of D1 of msCEACAM1a[1,4], CD2, CD4 and Bence-Jones protein REI. Each molecule is shown in Cα trace, with msCEACAM1a in cyan, CD2 in purple, CD4 in brown and REI in green, respectively. The uniquely convoluted conformation of the CC′ loop in msCEACAM1a[1,4] is striking. The sequence alignment of the CC′ loop regions of these four molecules are also shown using the same code. (2B) Stereo view of the exposed residues on the CFG face of D1 of msCEACAM1a[1,4]. The Cu. trace of the CC′ loop is highlighted. Displayed sidechains and carbohydrates are drawn in ball-and-stick style. (2C) Change the legend to “Electrostatic potential surface representation of the same view as (B). The electrostatic potential is colored blue for positive and red for negative, and was calculated in the absence of carbohydrates and solvent molecules. FIGS. 2A and B were prepared with MOLSCRIPT® (Krulis, 1991), and 2C, with GRASP® (Nicholls et al., 1991).

FIG. 3. A comparative view of structures of several virus receptors, including msCEACAM1a, receptor for murine coronavirus MHV; ICAM1, receptor for the major group of rhinoviruses; CD4, primary receptor for HIV; and CD46, receptor for measles virus. Shown here are only their N-terminal domains. Their key virus-binding motifs with uniquely topological features are also highlighted.

FIG. 4. Sequence alignment of D1 and D4 of murine CEACAM1 with corresponding domains of human CEA family members. Residues invariant throughout all sequences shown are colored yellow, whereas physico-chemically conserved residues (with no more than two exceptions) are colored blue. The β strands are shown underlined. (4A) D1 of murine CEACAM1a is aligned with D1 of murine CEACAM1b (upper panel), as well as the human CEA members found in the SWISSPROT database (lower panel). (4B) D4 of murine CEACAM1a is aligned with D2 of the same molecule (upper panel). The marks potential N-glycosylation sites. These sequences are compared with the A1, A2, A3 and B1, B2, B3 domains of human CEA, the gene product of CEACAM5 (lower panel).

FIG. 5. Topology diagram for D1 of msCEACAM1a with 0 strands shown as arrows. The diagram is colored according to the degree of variability in sequence of N-terminal domain for all available mammalian CEA molecules. The variability was measured using Shannon's entropy value (H) (Stewart et al., 1997). The least variable, or most conserved, residues (H<1) are colored green, whereas the most variable ones (H>2) are colored red. Those residues in between (1<H<2) are colored yellow. The difference in the degree of sequence conservation between the ABED and CFG faces is evident. On the ABED face, the glycan at Asn70 and the shielded hydrophobic residues are marked.

FIG. 6A and B. Backbone worm representation of the “parallel” interaction between the dyad-related msCEACAM1a[1,4] molecules seen in the crystal structure, prepared with GRASP® (Nicholls et al., 1991). (6A) Two monomers related by a crystallographic 2-fold axis are shown in blue and green, respectively. Carbohydrates are drawn in ball-and-stick style. (6B) Stereo picture of the close-up view across the dimer interface. Those sidechain involved in interactions are shown in ball-and-stick style.

### DETAILED DESCRIPTION OF THE PREFERRED EMBODIMENTS

The present invention is illustrated in further detail by the following non-limiting examples. Although the following descriptions re directed to preferred embodiments, namely a molecular model useful for designing compounds that modulate the interaction between the novel structure of the CC′ loop of the carcinoembryonic antigen cell adhesion molecule and other molecules (e.g. antibodies), as well as the various compounds that will satisfy this criteria, it should be understood that this description is illustrated only and is not intended to limit the scope of the invention.

The amino acid residues described herein are preferred to be in the “L” isomeric form. However, residues in the “D” isomeric form can be substituted for any L-amino acid residue, as long as the desired fractional property of immunoglobulin-binding is retained by the polypeptide. NH.sub.2 refers to the free amino group present at the amino terminus of a polypeptide. COOH refers to the free carboxy group present at the carboxy terminus of a polypeptide. In keeping with standard polypeptide nomenclature, J. Biol. Chem., 243:3552-59 (1969), abbreviations for amino acid residues are shown in the following Table of Correspondence:

TABLE OF CORRESPONDENCE

SYMBOL

| 1-Letter | 3-Letter | AMINO ACID |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| W | Trp | tryptophan |
| R | Arg | arginine |
| D | Asp | aspartic acid |
| N | Asn | asparagine |
| C | Cys | cysteine |

It should be noted that all amino-acid residue sequences are represented herein by formulae whose left and right orientation is in the conventional direction of amino-terminus to carboxy-terminus. Furthermore, it should be noted that a dash at the beginning or end of an amino acid residue sequence indicates a peptide bond to a further sequence of one or more amino-acid residues. The above Table is presented to correlate the three-letter and one-letter notations which may appear alternately herein.

A number of articles review computer modeling of drugs interactive with specific proteins, such as Rotivinen (1988, Acta Pharmaceutical Fennica '97. 159-166); Ripka (1988 New Scientist 54-57); McKinaly and Rossmann (1989, Ann. Rev. Pharmacol. Toxicol. 29: 111-122); Perry and Davies, OSAR; Quantitative Structure-Activity Relationships in Drug Design pp. 189-193 Alan R. Liss, Inc. 1989; Lewis and Dean (1989, Proc. R. Soc. Lond. 236: 125-140 and 141-162); and with respect to a model receptor for nucleic acid components, Askew, et al. (1989, J. Am. Chem. Soc. 111: 1082-1090). Other computer programs that screen and graphically depict chemicals are available from companies such as BioDesign, Inc. (Pasadena, Calif.), Allelix, Inc. (Mississauga, Ontario, Canada), and Hypercube, Inc. (Cambridge, Ontario).

Although described above with reference to design and generation of compounds which could alter binding, one could also screen libraries of known compounds, including natural products or synthetic chemicals, and biologically active materials, including proteins, for compounds which are inhibitors or activators.

Compounds identified via assays such as those described herein may be useful, for example, for treating any of the conditions disclosed herein that depend upon biological interactions of CEACAM1 or structurally related proteins. Assays for testing the efficacy of compounds identified in the cellular screen can be tested in animal model systems for such conditions. Such animal models may be used as test substrates for the identification of drugs, pharmaceuticals, therapies and interventions which may be effective in treating such conditions. For example, animal models may be exposed to a compound suspected of exhibiting an ability to ameliorate a condition mediated by CEACAM1 or related proteins at a sufficient concentration and for a time sufficient to elicit such an amelioration of condition-associated symptoms in the exposed animals. The response of the animals to the exposure may be monitored by assessing the reversal of symptoms associated with the condition, such as an autoimmune condition or a delayed hypersensitivity response to an antigen, or by assessing prevention of infection with a virus or bacterium that depends upon binding to CEACAM1 or structurally related proteins on host cell membranes. With regard to intervention, any treatments which reverse any aspect of such symptoms should be considered as candidates for human therapeutic intervention. Dosages of test agents may be determined by deriving dose-responsive curves, in accordance with standard practice.

According to still another aspect of the invention, low molecular weight compounds that inhibit the interaction between CEACAM1 or structurally related proteins and their natural ligands in the body or proteins of bacteria or viruses that use these molecules as receptors are provided. These compounds can be used to modulate the interaction or can be used as lead compounds for the design of better compounds using the above-described computer-based rational drug design methods.

As also described in U.S. Pat. No. 5,908,609, exemplary library compounds include, but are not limited to, peptides such as, for example, soluble peptides, including but not limited to members of random peptide libraries; (see, e.g., Lam, K. S. et al., 1991, Nature 354:82-84; Houghten, R. et al., 1991, Nature 354:84-86), and combinatorial chemistry-derived molecular libraries made of D-and/or L-configuration amino acids, phosphopeptides (including but not limited to, members of random or partially degenerate, directed phosphopeptide libraries; (see, e.g., Songyang, Z. et al., 1993, Cell 72: 767-778); antibodies (including, but not limited to, polyclonal, monoclonal, humanized, anti-idiotypic, chimeric or single chain antibodies, and Fab, F(ab), sub. 2 and Fab expression library fragments, and epitope-binding fragments thereof), and small organic or inorganic molecules. Other compounds which can be screened in accordance with the invention include but are not limited to small organic molecules that are able to gain entry into an appropriate cell and affect the interaction of CEACAM1 (or structurally related proteins in the CEA family) with its natural ligands in vivo or with bacteria or viruses. For example, the compounds of the invention that can be designed to satisfy the foregoing criteria include polypeptides and peptide mimetics. The peptide mimetic can be a hybrid molecule which includes both amino acid and non-amino acid components, e.g., the mimic can include amino acid components for the positively charged and negatively charged regions and non-amino acid (e.g., piperidine) having the same approximate size and dimension of a hydrophobic amino acid (e.g., phenylalanine) as the hydrophobic component.

In certain preferred embodiments, the screening assay is designed to identify agents which modulate the interaction of the CEACAM1 or structurally related protein with the viral spike glycoprotein or a bacterial adhesion molecule or outer membrane protein (referred to in the art as a heterophilic interaction) and not interfere with homophilic interactions (e.g., CEACAM1 binding to another CEACAM1 or structurally related molecule). In this manner, agents can be selected which advantageously affect only the interaction of CEACAM1 or structurally related proteins with bacteria or viruses without adversely affecting other natural cellular functions of these polypeptides. In these and other embodiments, the assays optionally involve the step of introducing the compound into an animal model of a condition mediated by the interaction of CEACAM1 or structurally related proteins and pathogenic bacteria or viruses and determining whether the compound prevents infection or alleviates the symptoms of the condition. At the same time, the natural cellular functions of CEACAM1 in cell adhesion, immune interactions, angiogenesis, etc. would be assayed to assure that these were normal, i.e., within pharmacological acceptable levels.

In general, the assay can be of any type, provided that the assay is capable of detecting the interaction of a CEACAM 1 or structurally related protein and a natural ligand. Preferably, the assay is a binding assay (e.g., an adhesion assay) which detects adhesion between the CEACAM1 or structurally related protein and the domain or polypeptide of the natural ligand that binds to CEACAM1 or related protein. Exemplary adhesion assays are described in the Examples. In general, such assays can be performed using cell-free or cell-based systems, e.g., the polypeptide components can be isolated or can be expressed on the surface of a cell. Additionally, or alternatively, the assay can be a signaling assay which detects signaling events following interaction of the ligand or domain of the ligand and the CEACAM1 (or related) protein or the ligand-binding domain of CEACAM1. In such instances, the signaling assay typically is a cell-based assay in which the CEACAM1 protein is expressed on a cell. In a cell signaling assay, a down-stream effect (e.g., a change in cytokine expression, enhanced expression of another gene) or altered expression of a receptor due to CEACAM1 binding to the ligand or the CEACAM1-binding domain of the ligand is detected, rather than detecting only the adhesion of these molecules to one another.

Regardless of the particular type of assay, in the some embodiments, the assays of the invention may utilize an isolated ligand for CEACAM1, unless the assay further involves the selection of a molecular library, which takes into account the information presented herein with respect to the approximately size and charge characteristics of prospective modulators of the interaction. In the latter instances, the CC′ loop of CEACAM1 or a domain of its natural ligand that binds to the CC′ loop of CEACAM1 may form part of a synthesized or recombinant polypeptide that may or may not be complexed to a marker polypeptide or molecule. The assays of the invention may utilize CEACAM1 protein which is complete or, alternatively, which contains CEACAM1 N-terminal domain (e.g., at least an isolated CC loop but not the entire 4 domain anchored CEACAM1 polypeptide sequence. The protein or peptide may be used in isolated form (e.g., immobilized to a solid support or as a soluble fusion protein as described in the examples) or expressed on the surface of a cell (e.g., an epithelial cell, an endothelial cell, or other cell genetically engineered to express the CEACAM1. The ligand polypeptide that binds to the CC′ loop of CEACAM1 (such as a viral spike glycoprotein, or bacterial outer membrane protein, or homophillic binding domain of CEACAM1) likewise may be used in isolated form or expressed on the surface of a cell.

As used herein in reference to a peptide, the term “isolated” refers to a cloned expression product of an oligonucleotide; a peptide which is isolated following cleavage from a larger polypeptide; or a peptide that is synthesized, e.g., using solution and/or solid phase peptide synthesis methods as disclosed in, for example, U.S. Pat. No. 5,120,830, the entire contents of which are incorporated herein by reference. Accordingly, the phrase “isolated peptides” embraces peptide fragments of CEACAM1 or its ligands as well as functionally equivalent peptide analogs (defined below) of the foregoing peptide fragments. As used herein, the term “peptide analog” refers to a peptide which shares a common structural feature with the molecule to which it is deemed to be an analog. A “functionally equivalent” peptide analog is a peptide analog which further shares a common functional activity with the molecule to which it is deemed an analog. Alternatively, the binding partners in the adhesion assays can be the particular ligands and receptors which mediate intercellular adhesion. For example, the binding of a lymphocyte, macrophage, polymorphonuclear cell or dendritic cell to an epithelial or endothelial cell may be mediated via the specific interaction of CEACAM1 and CEACAM1(on the epithelial cell). Accordingly, adhesion assays can be performed in which the binding partners are: (1) interacting cells (e.g. a lymphocyte and an epithelial cell); (2) a cell expression a ligand (e.g. an lymphocyte expressing CEACAM1 or a structurally related protein) and an isolated receptor (e.g. soluble recombinant CEACAM1) for the ligand; (3) an isolated ligand and a cell expressing the receptor for the ligand; and (4) an isolated ligand and its isolated receptor (e.g. viral spike protein). Thus, a high throughput screening assay for selecting pharmaceutical lead compounds can be performed in which, for example, (1) CEACAM1 is immobilized onto the surface of a microtiter well, (2) aliquots of a molecular library containing library members selected to accordance with the methods of the invention are added to the wells, 93) (labeled) cells expressing a ligand for CEACAM1 (e.g. lymphocytes) are added to the wells and (4) the well components are allowed to incubate for a period of time that is sufficient for the lymphocytes to bind to the immobilized CEACAM1. Preferably, the lymphocytes (or soluble CEACAM1-binding protein or peptide) are labeled (e.g., preincubated with Cr or a fluorescent dye) prior to their addition to the microtiter well. Following the incubation period, the wells are washed to remove non-adherent cells and the signal (attributable to the label on the remaining attached lymphocytes is determined. A positive control (e.g., a cell type that is known to bind to CEACAM1) on the same microtiter plate is used to establish maximal adhesion value. A negative control (e.g., soluble CEACAM1 added to the microtiter well) on the same microtiter plate is used to establish maximal levels of inhibition of adhesion.

The screening methods of the invention provide useful information for the rational drug design of novel agents which are, for example, capable of modulating an immune system response, or blocking viral or bacterial infection. In addition to the above-noted computer model programs, exemplary procedures for reational drug design are provided in Saragovi, H. er al., (1992) Biotechnology 10:773; Haber E. (1983) Biochem, Pharmacol. 32(13(:1967; and Connolly Y., (1991) Methods of Enzym9logy 203, Ch. 29 “Computer-Assisted Rational Drug Design”: pp 587-616, the contents of which are incorporated herein by reference.

Thus, knowledge of the structure (primary, secondary or tertiary) of naturally occurring ligands and receptors can be used to rationally choose or design molecules which will bind with either the ligand or receptor. In particular, knowledge of the binding regions of ligands and receptors can be used to rationally choose or design compounds which are ore potent than the naturally occurring ligands in eliciting their normal response or which are competitive inhibitors of the ligand-receptor interaction.

Once rationally chosen or designed and selected, the library members may be altered, e.g., in primary sequence, to produce new and different peptides. These fragments may be produced by site-directed mutagenesis or may be synthesized in vitro. These new fragments may then be tested for their ability to bind to the receptor or ligand and, by varying their primary sequences and observing the effects, peptides with increased binding or inhibitory ability can be produced. For example, improved compounds which modulate the interaction of a cell adhesion assay can be made by making conservative amino acid substitutes in peptides (e.g., Formula I) that are designed to fit in the active site defined by the docking model disclosed herein. As used herein, “conservative amino acid substitution” refers to an amino acid substitution which does not alter the relative charge or site characteristics of the peptide in which the amino acid substitution is made.

It will be appreciated by those skilled in the art that various modifications of the foregoing peptide analogs can be made without departing from the essential nature of the invention. Accordingly, it is intended that peptides which include conservative substitutions and couples proteins in which a peptide of the invention is coupled to a solid support (such as a polymeric bead), a carrier molecule such as keyhole limpet hemocyanin), a toxin (such as ricin) or a reporter group (such as radiolabel or other tag), also are embraced within the teachings of the invention.

The screening assays of the invention are useful for identifying pharmaceutical lead compounds in molecular libraries. A “molecular library” refers to a collection of structurally-diverse molecules. Molecular libraries can be chemically-synthesized or recombinantly produced. As used herein, a “molecular library member” refers to a molecule that is contained within the molecular library. Accordingly, screening refers to the process by which library molecules are tested for the ability to modulate (i.e., inhibit or enhance) interaction between a CEACAM1 or structurally related protein and a naturally occurring ligand, or a viral protein or bacterial protein and an antibody specific for CEACAM1, particularly the biologically active CC′ loop which has the unique structure described herein. As used herein, a “pharmaceutical lead compound” refers to a molecule example, screening assays are useful for assessing the ability of a library molecule to inhibit the binding of a CEACAM1 ligand (or an polypeptide derived from CEACAM 1 or structurally related protein) to a natural ligand.

Libraries of molecularly diverse molecules can be prepared used chemical and/or recombinant technology. Such libraries for screening include recombinantly produced libraries of fusion proteins. An exemplary recombinantly produced library is prepared by ligating fragments of CEACAM1 or related protein into, for example, the pGEX2T vector (Pharmacia, Piscataway, N.H.). This vector contains the carboxy terminus of glutathion S-transfersse (GST) from Schistosoma japonicum. Use of the GST-containing vector facilitates purification of GST-polypeptide fusion proteins from bacterial lysates by affinity chromatography on glutathione sepherose. After elution from the affinity column, the fusion proteins are tested for activity by, for example, subjecting the fusion protein to the screening assays disclosed herein. Fusion proteins which inhibit binding between CEACAM 1 expressing cells are selected as pharmaceutical lead compounds and/or to facilitate further characterization of the portion of the lead compound which the blocks homophilic binding

The methods of the invention are useful for identifying novel compounds that are capable of modulating a mucosal immune response in vivo. Accordingly, the invention further provides a pharmaceutical preparation for modulating a mucosal immune response in a subject is provided. The composition includes a pharmaceutically acceptable carrier and an agent that inhibits interaction (e.g., adhesion) between an CC′ domain and CEACAM1. In particularly preferred embodiments, the agent inhibits homophlic adhesion between a CEACAM1-expressing cells. The agent (e.g., the above-described peptide) is present in a therapeutically effective amount for treating the immune response or treating or preventing viral or bacterial infection. Thus, in a related aspect, the invention also provides a method for modulating the mucosal immune response of a subject. The method involves administering to the subject a pharmaceutical composition containing the above-described agents for inhibiting adhesion between a CEACAM1-expressing cells. In addition the same compounds can be tested for the ability to inhibit or treat bacterial or viral infections of microbes that use CEACAM1 as receptors.

In general, the therapeutically effective amount is between about 1 mg and about 100 mg/kg. The preferred amount can be determined by one of ordinary skill in the art in accordance with standard practice for determining optimum dosage levels of the agent. The compounds are formulated into a pharmaceutical composition by combination with an appropriate pharmaceutically acceptable carrier. For example, the compounds may be used in the form of their pharmaceutically acceptable salts, or may be used alone or in appropriate association, as well as in combination with other pharmaceutically active compounds. The compounds may be formulated into preparations in solid, semisolid liquid, or gaseous form such as tablets, capsules, powders, granules, ointments, solutions, suppositories, inhalants and injections, in usual ways for oral, parenteral, or surgical administration. Exemplary pharmaceutically acceptable carriers are described in U.S. 5,211,657, the entire contents of which patent are incorporated herein by reference. The invention also includes locally administering the composition as an implant.

### EXAMPLE 1

Protein Expression and Purification

Nucleotide sequences encoding the first 236 amino acids of murine CEACAM1a[1,4] including the natural 34 aa long signal sequence were amplified by PCR using an oligonucleotide that added an XbaI site in frame at the 3′ end. This DNA was ligated in frame into a previously described construct encoding a thrombin cleavage peptide followed by six histidine residues and a stop codon (Zelus et al., 1998), and inserted into the pShuttle CMV vector (He et al., 1998). This construct was inserted into the pAd-Easy adenovirus vector, and adenoviruses that contained the cDNA were plaque purified and amplified in 293 cells as previously described (He et al., 1998). Lec-CHO cells stably transfected with CAR, the Coxsackie/adenovirus receptor were transduced with the CEACAM1a[1,4]-containing adenovirus. The soluble, his-tagged murine CEACAM1a[1,4] protein from the supernatant medium was purified by nickel affinity chromatography on a Pharmacia HiTrap chelating column, and eluted with imidazole. Fractions containing the protein were identified by immunoblotting with polyclonal rabbit antibody directed against murine CEACAM1a, and the pooled fractions were dialyzed against 25 mM Tris buffer, pH 9.0, with 5% glycerol. The protein was further purified by ion exchange chromatography on a HQ20 (Poros) column and eluted in a sodium chloride gradient. Fractions containing the protein were pooled, dialyzed against 25 mM TRIS pH (7.6), 150 mM NaCl, 5% glycerol, and stored at −80° C. The purity of the proteins was determined by silver staining of SDS-PAGE gels and by Western blotting with anti-CEACAM1a antibody. The medium of 40 T150 flasks of adenovirus transduced lec-,CAR+CHO cells yielded approximately 0.5 to 1 mg of purified msCEACAM1a[1,4] protein.

### EXAMPLE 2

Crystallization and X-Ray Data Collection

Single crystals of msCEACAM1a[1,4] were grown from a crystallization buffer containing 10% PEG 8000, 0.2 M magnesium acetate and 0.1 M cacodylate at pH 6.4 using the vapor-diffusion hanging drop method. For data collection at cryogenic temperature, the crystals were treated with a cryoprotectant solution (25% glycerol, 10% PEG 8000 and 0.1 M cacodylate), then frozen and stored in liquid nitrogen. Platinum derivatives were prepared by soaking the crystals overnight in the same cryo-protectant solution containing 0.5 mM K₂PtBr₄.

X-ray diffraction data were collected from pre-frozen crystals at APS SBC 19ID at a temperature of 100° K. A native crystal diffracted to a resolution of 3.32 Å, with one molecule in one asymmetric unit. A multi-wavelength anomalous diffraction (MAD) data set of the platinum derivative was obtained to a resolution of 3.85 Å. All the raw data were indexed and reduced with HKL2000 (Otwinowski and Minor, 1997)(Table I).

### EXAMPLE 3

Structure Determination and Refinement

The msCEACAM1a[1,4] structure was solved using the MAD phases in combination with molecular replacement (MR). Using programs in the CCP4 suite (CCP4, 1994), one Pt binding site was identified in one asymmetric unit in both difference and anomalous difference Patterson maps. Heavy atom parameters were refined at 4 Å resolution with the program MLPHARE in CCP4 suite, and an additional platinum site was identified. Phase extension was performed using the native data set to 3.32 Å by solvent flattening and histogram matching with DM. The resulting phases were used to carry out a phased molecular replacement with ROTPTF on the Bronx X-ray server for the two separate domains. The N-terminal domains of CD2 (PDB code 1HNF) and human Fc-γ receptor III (PDB code 1E4J) were used as search models for the D1 and D4 domains of msCEACAM1a[1,4], respectively. The model was traced with XtalView (http://www.scripts.edu/pub/dem-web)on the basis of the MAD phases, using the MR solutions as a guideline.

After cycles of model building using program O (Jones et al., 1991) and refinement, the final model was refined at 3.32 Å resolution to an R_{free} factor of 32.9% and R_{work} of 29.5% (Table I) using the Xplor (Brunger, 1992). At 1.5σ contour level (σ=0.125 e/Å³) in 2Fo-Fc map, there was continuous density for the main chain backbone. The final model contains 203 residues (from Glu1 to Thr203) and a total of 6 sugar residues associated with four of the five potential glycosylation sites. There was no visible electron density beyond residue Thr203 where more than a dozen residues including a his-tag are present in the expression construct. These C-terminal residues are apparently disordered. The current model also includes a total of 26 water molecules. Some of the densities assigned to solvent molecules around the end of glycans might be from partially disordered branched sugar residues.

### EXAMPLE 4

Molecular structure of msCEACAM1a[1,4]

The msCEACAM1a[1,4] protein analyzed contains the 202 extracellular amino acids of the naturally expressed CEACAM1a[1,4] protein plus a six histidine-tag connected to the carboxy-terminus by a thrombin cleavage peptide. This soluble murine CEACAM1a[1,4] protein has strong virus neutralization activity at 37° C., pH 7.2, and readily induces an irreversible conformational change in the MHV-A59 spike glycoprotein under these conditions (Zelus et al., 1998). The his-tagged protein was expressed by an adenovirus vector in the Chinese hamster ovary Lec3.2.8.1 (CHO lec-) cell line that stably expresses recombinant CAR, the receptor for Coxsackie B and adenoviruses (Bergelson et al., 1997; Stanley, 1989; Zelus et al., 1998). These cells were readily transduced by the adenovirus vector, and they produce proteins with more homogeneous glycans than normal CHO cells. Analysis of the protein secreted by the lec-, CAR+CHO cells led to the final refined model for the structure of msCEACAM1a[1,4]. The structure was determined using the multi-wavelength anomalous diffraction (MAD) phases in combination with molecular replacement (MR).

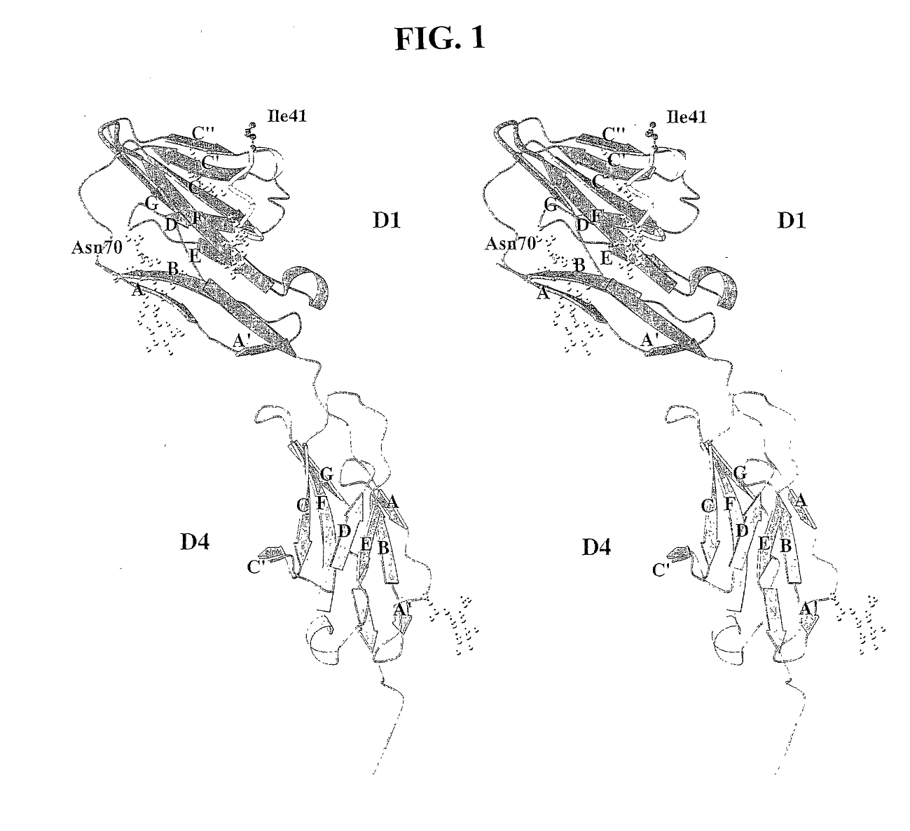

FIG. 1 shows the ribbon diagram of the molecular structure of soluble murine msCEACAM1a [1,4]. The two Ig-like domains of msCEACAM1a[1,4] are arranged in tandem. When the membrane proximal domain (D4) was oriented vertically as if it were perpendicular to the cell membrane, the virus-binding domain (D1) had a bending angle of about 600 from the vertical, with its A′GFCC′C″ β sheet (called CFG face hereafter) facing upwards, away from the cell membrane (FIG. 1). The rotation angle between D1 and D4 is about 170°, which places the CFG face of D4 on the opposite side of the molecule from the CFG face of D1, Other IgSF proteins on the cell surface have this orientation (Wang and Springer, 1998). Although there are five potential N-linked glycosylation sites on this protein, the crystal structure showed that only four of these sites are utilized: three in D1, and one in D4. One or more sugar moieties were clearly seen at each of these sites (FIG. 1), but no electron density was visible to indicate the presence of a possible glycan at Asn161 in the Asn-Asn-Ser motif in the DE loop of D4. The only observed glycan in D4 is at Asn119 (FIG. 1) near the bottom of the molecule, pointing downward toward the cell membrane. This glycan may play a role in holding the rod-like molecule erect on the membrane as shown for CD2 (Jones et al., 1992), ICAM-2 (Casasnovas et al., 1997), and CD4 (Wu et al., 1997).

The N-terminal domain (D1) of msCEACAM1a[1,4] belongs to the V set Ig-like fold. Within the IgSF, the CEA family and the CD2 family are uniquely in that their N-terminal domains lack the inter-sheet disulfide bond between β strands B and F that is conserved in the N-terminal domains of other IgSF members. In the DALI search for structures homologous to D1 of msCEACAM1a[1,4] using the web site (http:H/www2.ebi.ac.uk/dali/), D1 of CD2 was one of the top hits. There are, however, three important structural elements that distinguish D1 of msCEACAM1a[1,4] from CD2-D1. One striking feature of D1 of msCEACAM1a[1,4] is its uniquelyly structured, prominently protruding CC′ loop (highlighted in FIG. 1) that points upwards. The uniquely and intricate structure of the CC′ loop will be described in detail below. D1 of msCEACAM1a[1,4], like other V set Ig-like folds, retains a salt bridge between an arginine (Arg64) at the beginning of the D strand and an aspartate (Asp82) at the beginning of the F strand. This salt bridge may help to strengthen the interactions between the two anti-parallel β sheets of D1. By contrast, CD2-D1 does not have a salt bridge between the β sheets (Jones et al., 1992). Another difference between the D1s of msCEACAM1a[1,4] and CD2 is found at the A-A′ kink. As a structural hallmark in both V set and I set Ig folds, the A strand in one sheet runs midway through the domain, and then crosses over to join the opposite sheet, becoming the A′ strand. This may stabilize the membrane-distal domain that is usually the site for ligand binding (Wang and Springer, 1998). The amino acid at the kink position is usually a cis-proline. In D1 of msCEACAM1a[1,4], the A′ strand is significantly shorter than that of most other Ig-like molecules, whereas D1 of CD2 and some other CD2 family members have a relatively long A′ strand with no A strand at all. These features might reflect differences in the biological functions of CD2 and CEACAM1a.

Structural analysis shows that the C-terminal domain (D4) of msCEACAM1a[1,4] falls into the I1 set category (Harpaz and Chothia, 1994; Wang and Springer, 1998), rather than the C2 set as widely thought. Compared to the I set Ig-like domains of most other IgSF members, D4 of msCEACAM1a[1,4] has an unusually long CD loop of 10 residues (amino acids 146-155). The long CD loop in D4 of msCEACAM1a[1,4] is probably quite stable because it has a β-turn at each end (including the 2 residue C′ strand) and Leu150 and Leu152 in the middle of the loop point inward, joining the molecule's hydrophobic core.

msCEACAM1a[1,4] has a linker between D1 and D4. The last residue of D1 is His107, and the A strand of the following domain D4 starts at Phe114. The peptide segment in between does not appear to have mainchain-mainchain hydrogen bonds to the D4 domain. No significant interactions were observed between D1 and D4. The surface buried area between these two domains is 530 Å², with a 1.7 Å probe. These observations indicate that the D1-D4 junction of msCEACAM1a[1,4] is quite flexible.

### EXAMPLE 5

The Uniquely CC′ Loop of the N-Terminal Domain is an MHV-Binding Site

Both the spike glycoprotein of MHV virions and MAb-CC1, a monoclonal antibody to murine CEACAM1a that blocks the binding of the virus to the receptor, were shown to bind to D1 of murine CEACAM1a (Dveksler et al., 1993b). Mutational analyses of murine CEACAM1a show that the peptide segments between amino acids 38 and 43 (Rao et al., 1997) or between amino acids 34 and 52 (Wessner et al., 1998) are involved in binding to the MHV spike glycoprotein, in virus receptor activity and binding of MAb-CC1. The structure for msCEACAM1a[1,4] defined in the present invention shows that this virus binding region is in the CC′ loop and the C′ strand.

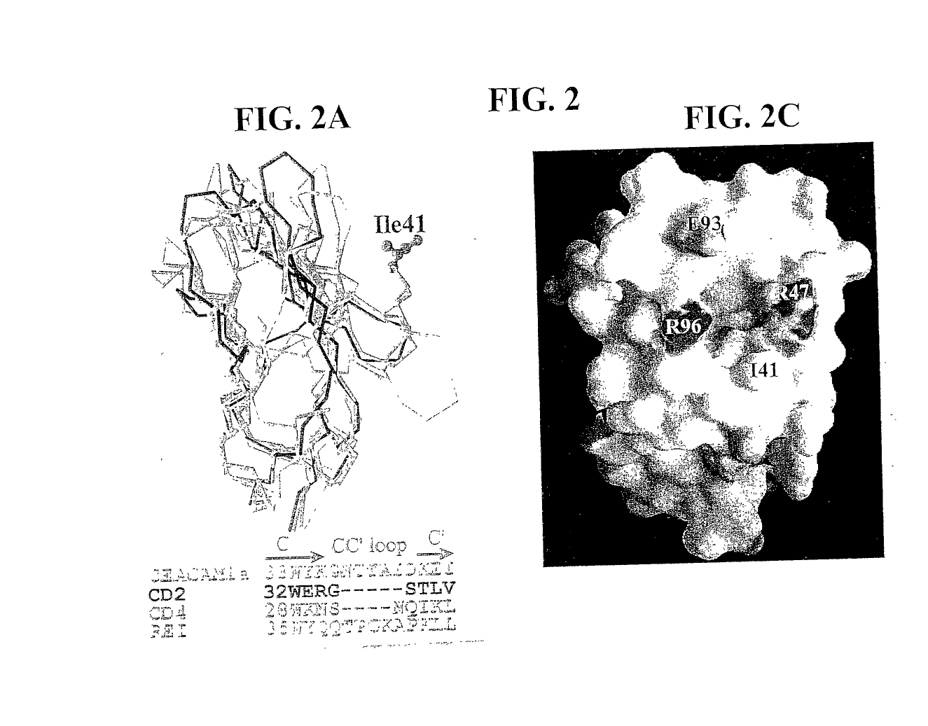

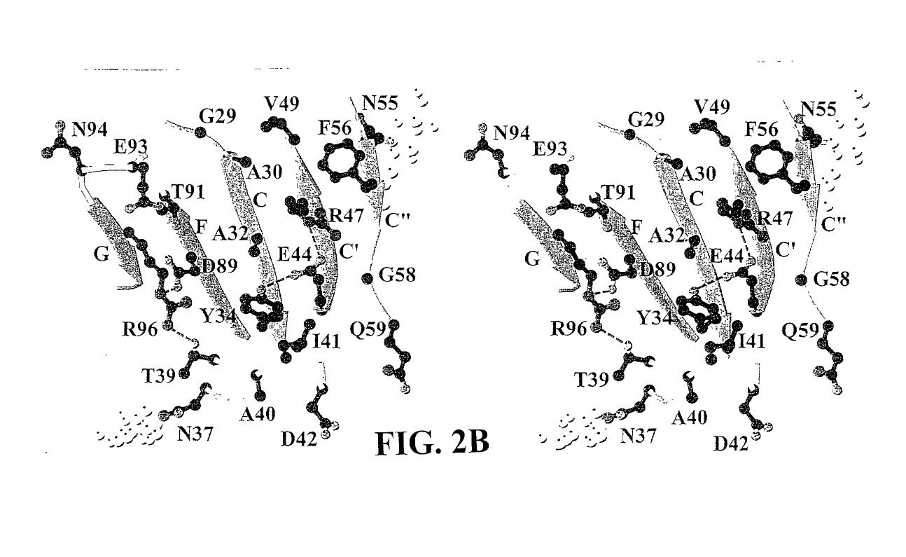

Compared to the N-terminal domains of other IgSF members, D1 of msCEACAM1a[1,4] has an unusual CC′ loop, highlighted in yellow in FIG. 1. This structure could not have been predicted based on the knowledge of the amino acid sequence in this region. FIG. 2A shows an overlay onto D1 of msCEACAM1a[ 1,4] of the N-terminal domains of three other representative IgSF proteins, CD2 (Jones et al., 1992), CD4 (Wang et al., 1990), and Bence-Jones protein REI (Epp et al., 1975), a typical variable domain of an antibody. The N-terminal domains of both CD2 and CD4 have shorter CC′ loops than that of msCEACAM1a[1,4] and REI. Although the CC′ loops of D1 of REI and msCEACAM1a[1,4] are the same length, that of REI is only slightly curved, while the CC′ loop of msCEACAM1a[1,4] remarkably folds back onto the CFG face.

The convoluted conformation of the CC′ loop in D1 of msCEACAM1a[1,4] is uniquely among IgSF molecules. The loop, from Lys35 to Glu44, is well structured (FIG. 2B) and probably maintained in a rigid conformation. Within the C terminal portion of the loop (residues 40 to 44), two mainchain hydrogen bonds form one and a half turns of a 3₁₀ helix. On the N-terminus of the CC′ loop, Thr38 forms a hydrogen bond with the carbonyl oxygen of Lys35, The mid portion of the CC′ loop makes close contact with the CFG face in two ways (FIG. 2B). Particularly interesting is the packing of two consecutive planar peptide groups on the loop, Thr39-Ala40 and Ala40-Ile41, against the aromatic ring of Tyr34 on the C strand. In addition, a bidentate hydrogen bond from the side-chain of Glu44 to side-chains of this Tyr34 and Arg47 helps to hold the aromatic ring in place for its interactions with the peptide groups. An additional hydrogen bond between the sidechains of Thr39 and Arg96 would also hold the CC′ loop toward the β sheet. Although a tyrosine equivalent to Tyr34 is conserved in the variable domains of most antibody light chains, nevertheless the CC′ loop in antibodies assumes a β hairpin structure (see REI in FIG. 2A) probably because the conserved Pro-Gly sequence motif of antibodies (FIG. 2A) favors a sharp turn at the tip of the loop. This might prevent the CC′ loop of REI from assuming a convoluted conformation like that seen in D1 of msCEACAM1a[1,4].

In D1 of msCEACAM1a[1,4], the consequence of the folding back of the highly structured CC′ loop against the CFG face is to cause the sidechain of Ile41 at the center of the loop to be prominently exposed, pointing away from the membrane (FIGS. 1 and 2A). Mutational evidence suggests that the Thr38-Thr39-Ala40-Ile41 sequence motif in murine CEACAM1a[1,4] is important for binding to the MHV spike glycoprotein (Wessner et al., 1998). Two glycans, one at Asn37 and the other at Asn55, flank this important virus-binding motif (FIGS. 1 and 2B), which might help delineate the region for viral spike glycoprotein docking. Based on the structural data presented here, Ile41 is considered to be the energetic “hot spot” for binding to the MHV spike. A widely accepted model for the interaction of cell surface receptors with their ligands is that a central hydrophobic contact provides the major binding energy, while surrounding hydrophilic interactions contribute the specificity of binding (Clackson and Wells, 1995). This also appears to be the case for receptor/virus interactions as shown for binding of gp120 glycoprotein of HIV-1 to CD4 (Kwong et al., 1998). FIGS. 2B and 2C show a view looking from above down upon the CFG face of D1 of msCEACAM1a[1,4] which is likely to be the surface accessible to the MHV virus spike protein. The protruding hydrophobic Ile41 is surrounded by a number of surface-exposed charged residues, including Asp42, Glu44, Arg47, Asp89, Glu93, and Arg97. Ile41 might insert into a hypothetical hydrophobic pocket in the viral spike glycoprotein, and charged residues that surround the pocket could stabilize the MHV binding interaction and contribute to virus binding specificity. No structures are yet available for any coronavirus spike glycoproteins. Strains of MHV that differ in virulence and tissue tropism show considerable variation in the amino acid sequences of their S glycoproteins, yet all MHV strains tested can use murine CEACAM1a as a receptor. The observation that there is no single anti-S MAb that blocks infection by all strains of MHV (Talbot and Buchmeier, 1985) supports the idea that murine CEACAM1a may bind to a conserved pocket in S that is not accessible to antibody. The protruding Ile41 and the charged residues that surround it on the surface of the virus receptor are targets for further mutational analyses.

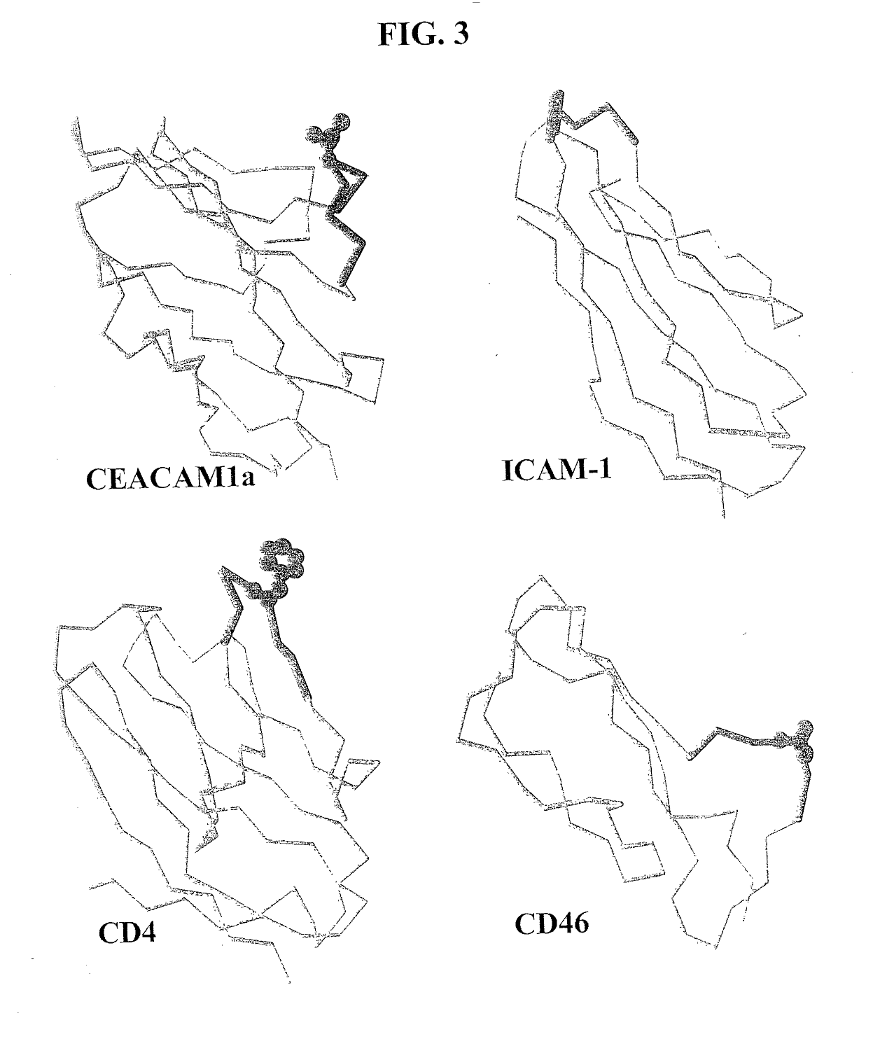

Cell adhesion molecules might be particularly suitable candidates for virus binding because their physiologic ligand/receptor binding affinities are very low, and adhesion is an avidity driven process. Uniquely exposed surface features of the cell adhesion molecules are Ito selected for virus binding. FIG. 3 compares the virus-binding domain of msCEACAM1a[1,4] with those of several other virus receptors with the key virus-binding elements highlighted. The projecting Ile41 on the uniquely CC′ loop of D1 of msCEACAM1a[1,4] is the key topological feature for MHV binding. In CD4, the key HIV gp120-binding Phe43 is located at the protruding ridge-like C′C″ corner of D1 (Wang et al., 1990). This structural element inserts into a recess in the surface of HIV gp120 (Kwong et al., 1998). Compared to most IgSF members, ICAM-1, the receptor for the major group of rhinoviruses, has a uniquely, tapering tip that inserts into the narrow “canyon” on the rhinovirus surface where the conserved receptor-binding epitopes lie hidden from immune recognition (Kolatkar et al., 1999). The measles virus receptor CD46 belongs to the complement control protein (CCP) superfamily. The center of the virus-binding epitope of CD46 is a well-structured, protruding DD′ loop consisting of a small group of hydrophobic residues with the key Pro39 extending furthest out (FIG. 3) (Casasnovas et al., 1999). Thus, uniquely protruding hydrophobic residues on cell adhesion molecules might be prime targets for virus binding.

### EXAMPLE 6

MHV Receptor Activities of Murine CEACAM Isoforms, Chimeras and Mutants

The various natural isoforms of the murine CEACAM1a, CEACAM1b and CEACAM2 glycoproteins differ markedly in their virus binding, neutralization and virus receptor activities (Dveksler et al., 1993a; Gallagher, 1997; Ohtsuka et al., 1996; Zelus et al., 1998). A series of soluble or anchored mutant murine CEACAM proteins with various point mutations, deletions, or domain exchanges with other CEA-related glycoproteins has been tested for virus binding and receptor activities (Rao et al., 1997; Wessner et al., 1998). Several observations were made. MHV-A59 and soluble spike protein bound better to D1 of murine CEACAM1a from MHV susceptible mice than to CEACAM1b from MKV-resistant mice. Soluble murine CEACAM1b[1-4] had 4 to 10 fold less virus neutralization activity for MHV-A59 than msCEACAM1a[1-4]. The msCEACAM1b[1-4] failed to neutralize the neurotropic JHM strain of MHV, and msCEACAM1b[1,4] failed to neutralize either MHV-A59 or MHV-JHM(Zelus et al., 1998). While the naturally occurring 2 domain CEACAM1a[1,4] isoform neutralized MHV-A59 nearly as well as the 4 domain isoform CEACAM1a[1-4], a carboxyl terminal deletion protein consisting of D1 and D2 (CEACAM1a[1,2]) had only minimal MHV-A59-neutralizing activity. Thus, there is virus strain specificity in the interactions of MHV with various CEACAM1 proteins, and regions of CEACAM1 outside of the virus-binding domain (D1) can affect virus-receptor activity.

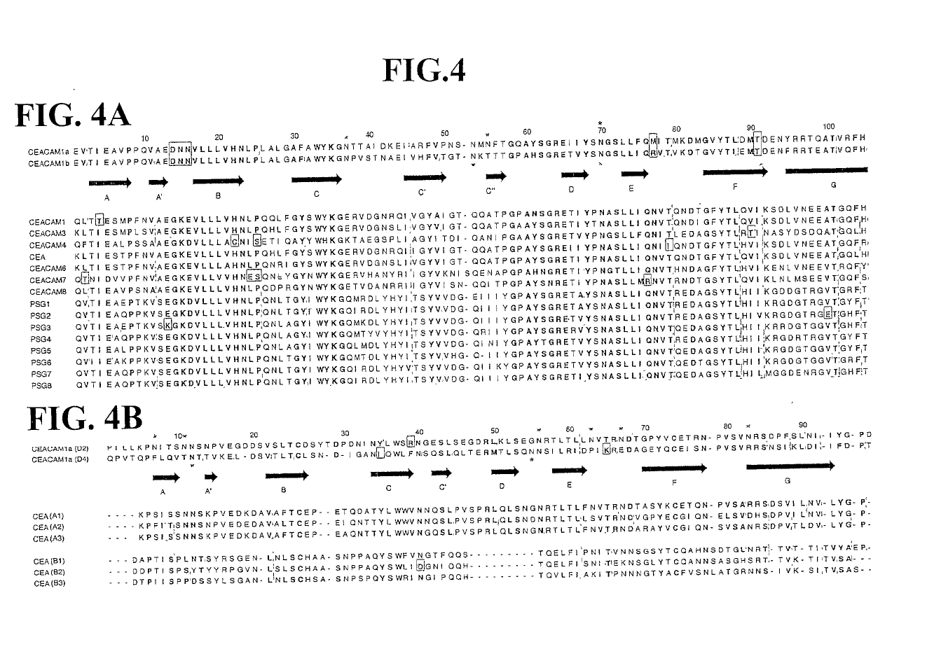

The amino acid sequences of murine CEACAM1a and CEACAM1b differ, principally in the N-terminal, virus-binding domain (Dveksler et al., 1993a). The lengths of the 1a and 1b proteins are the same, and all of the structurally important residues are the same or similar. The overall folding of murine CEACAM1b isoforms is therefore believed to be the same as or similar to that of the corresponding CEACAM1a isoforms. FIG. 4A (upper panel) shows the sequence alignment of D1 from murine CEACAM1a and CEACAM1b with β strands underlined. The most extensive differences between CEACAM1 a and 1b are in the peptide segment from the virus-binding CC′ loop to the end of the C″ strand. In D1 of CEACAM1b, residue Ile41 is replaced by a threonine, which may account for its low virus binding activity relative to 1-CEACAM1a.

Without the important Ile41, the question explored was why can murine CEACAM1b[1-4] serve as an MHV receptor. Comparison of the sequences in the CC′ loop region of D1 of CEACAM1a and 1b (FIG. 4A, upper panel) reveals two differences worthy of particular attention. Both Ile41 (Thr4l in CEACAM1b) and Thr39 (Val in CEACAM1b) are prominently exposed in the CC′ loop (FIG. 2B). In CEACAM1b, Pro38 replaces Thr38 of CEACAM1a and may change the conformation of the CC′ loop in CEACAM1b so that the projecting Val39 might serve as a virus-binding hotspot as Ile41 does for CEACAM1a, though to a lesser extent. Moreover, CEACAM1b lacks the glycosylation site at Asn37 of CEACAM1a due to the replacement of the N37TT sequence motif in CEACAM1a with N37PV. These differences in amino acid sequence and glycosylation probably also affect how spike proteins from various MHV strains dock on the different CEACAM receptor proteins, resulting in differences in receptor utilization, tissue tropism and virulence among the virus strains.

The carboxy-terminal deletion mutant msCEACAM1a[1,2] has very little virus neutralization activity, while the soluble form of the naturally occurring murine CEACAM1a[1,4] isoform neutralizes virus as well as the msCEACAM1a[1-4] isoform (Zelus et al., 1998). Analysis of the sequence alignment of domains 2 (D2) and 4 (D4) of CEACAM1a reveals two major differences (FIG. 4B, upper panel). The BC loop of D2 is two residues longer than that of D4, and D2 has four more potential N-glycosylation sites than D4 (marked with * in FIG. 4B). The longer BC loop of D2 and the possible glycan attached to Asn192 at the beginning of the G strand of D2 may both restrict inter-domain flexibility between D1 and D2 in msCEACAM1a[1,2] in comparison to the junction between D1 and D4 in msCEACAM1a[1,4]. Moreover, the present invention model building suggests that there is a hydrogen bond between His 107 of D1 and Asn141 of D2, while no such hydrogen bond is possible at this site in the junction of D1 and D4. All of these structural differences could cause the D1-D2 junction to be less flexible than the highly flexible junction between D1 and D4 revealed by X-ray crystallography. In CEACAM1a[1,2] on the cell membrane, the limited flexibility at the D1-D2 junction might make it more difficult for a virus to attach. The four domain isoform CEACAM1a[1-4] has two more interdomain junctions than the truncated CEACAM1a[1,2] protein, and may therefore be more flexible.

### EXAMPLE 7

Predicted Structures of CEA Family Members and Conservation of Glycan-Shielded Surface Hydrophobic Patch in the N-Terminal Domain

CEA family members are all composed of several Ig-like domains in tandem. Following the N-terminal domain, two similar types of domains, called A and B, alternate along the chain. For example, CEA (CD66e), encoded by the CEACAM5 gene, has the N-A1-B1-A2-B2-A3-B3 domain structure (Hammarstrom, 1999).

Blast search (http://www.ncbi.nlm.nih.gov/BLAST/) of D1 of murine CEACAM1a found sequences of N-terminal domains of all mammalian CEA members. Five residues appear to be absolutely conserved: Trp33, Arg64, Leu73, Asp82 and Tyr86 (FIG. 4A, lower panel). No significant deletions or insertions were found in D1 of human CEA-related proteins, except for a few cases in which the length of the C′C″ loop varied slightly. Like D1 of murine CEACAM1a, the N-terminal domains of all members of the CEA family shown in FIG. 4A can be classified as V set Ig-like fold.(Bates et al., 1992). This is determined by these key conserved structural features (Chothia et al., 1998): Pro8 at the A-A′ kink point; Trp33 on the C strand that acts as the center of a hydrophobic core; a salt bridge between Arg64 and Asp82; and the tyrosine-corner motif (Hemmingsen et al., 1994) D*G*Y86 at the beginning of the F strand.

One of the newly recognized, highly conserved structural features of msCEACAM1a[1,4] that appears to be uniquely to CEA family members (listed in FIG. 4A) is the glycosylation site at Asn70, on the opposite side of D1 from the proposed virus-binding surface (FIG. 1). In the crystal structure of msCEACAM1a[1,4], the glycan at Asn70 is better ordered than other glycans. Beneath the presumably large glycan at Asn70 lies a group of hydrophobic residues, including Val7 and Pro8 of the A strand, Leu18 and Leu20 of the B strand, Leu74 of the E strand, and probably also Tyr68 and Ile66 of the D strand. The area covers about 650 Å. The glycan at Asn70 appears to stabilize the protein by preventing the exposure of this large surface hydrophobic patch. Most of these protected amino acid residues are either invariant (Pro8 and Leu18) or very conserved (Leu20 , Tyr68 and Leu74) among CEA proteins (FIG. 4A). This is the first example of a three-dimensional structure consisting of a large, glycan-shielded surface hydrophobic patch that is conserved in a protein family. This structural feature is believed to have biological significance in the CEA family.

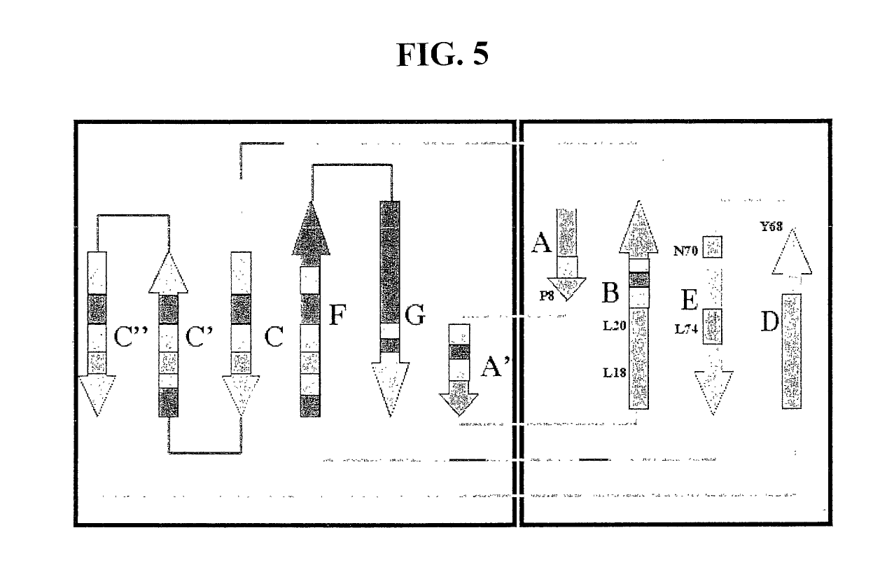

To assess the pattern of sequence conservation for all members of the mammalian CEA family in the SWISSPROT database, the variability in sequence using Shannon's entropy (Stewart et al., 1997) was calculated. FIG. 5 shows a topology diagram of D1 of msCEACAM1a[1,4] coded to indicate the relative degree of conservation of residues calculated for 42 CEA family members. A striking difference was discovered in the extent of amino acid conservation between the two faces of D1 among CEA family members. The ABED face containing the glycan-shielded hydrophobic patch is much more conserved than the CFG face. The CFG faces of the N-terminal domains of IgSF proteins are frequently used for cell surface recognition (Stuart and Jones, 1995; Wang and Springer, 1998). The variability in this face among CEA members is considered to their uniquely binding specificities.

In the lower panel of FIG. 4B, the sequences of the six A and B type domains of the human CEA protein are aligned with D2 and D4 of murine CEACAM1a. The three A type domains of human CEA, and probably the A domains of other CEA members as well, are structurally very homologous to D4 of murine CEACAM1a, an I1 set of Ig-fold. The B type domains of human CEA appear to have no D strand, but probably a C′ strand that directly connects to the E strand, as observed for 12 set of Ig-fold (Wang and Springer, 1998). Both I1 and I2 sets differ from the C set by having the A-A′ kink, and they are distinct from the V set in not having the C″ strand (Wang and Springer, 1998). In summary, data suggests that the general architecture of all CEA family members consists of a V set N-terminal domain followed by alternating I1 and I2 set Ig-like domains.

### EXAMPLE 8

The CC′ and FG Loops of the N-Terminal Domains of Various CEA Family Members Role in the Mediation of Biologically Important Molecular Interactions

The structure of murine CEACAM1a can be used to elucidate other molecular interactions of CEA family members including bacterial binding, immunomodulation, and homophilic and heterophilic adhesion.

Certain human CEA family members are subverted as receptors for bacterial pathogens including Hemophilus influenzae, Neisseria meningitidis and Neisseria gonorrhoeae. The N-terminal domains of many human CEA members are recognized by multiple Opa (opacity-associated) proteins on the surface of pathogenic strains of Neisseria (Bos et al., 1999; Virji et al., 1999). Homologue scanning mutagenesis revealed that Phe29, Ser32 and Gly41 (and to a lesser extent Gln44) of CEA (CD66e) are required for maximal Opa protein binding activity (Bos et al., 1999). Tyr34 and Ile91 (and to a lesser extent Val39 and Gln89) of human CEACAM1 (CD66a) are critical residues for most Opa protein interactions (Virji et al., 1999). Since the N-terminal domains of CEA and human CEACAM1 are the same length as that of murine CEACAM1a (FIG. 4A), FIG. 2B was used to show that the Neisseria-binding residues on CEA and human CEACAM1 are on the C strand through the CC′ loop and on the F strand. Val39 and Gly41 of human CEACAM1 and CEA, respectively (corresponding to Thr39 and Ile41 in msCEACAM1a[1,4], FIG. 2B) are on the tip of the CC′ loop. If the CC′ loops of CEA and CEACAM1 were as flat as that of the Bence-Jones protein REI (FIG. 2A), then Val39 and Gly41 would not be close enough to other important Opa-binding residues to form an integrated binding site. This may explain why the Y34A mutation of human CEACAM1 abrogated binding of the majority of Opa proteins (Virji et al., 1999), since the aromatic ring of this conserved Tyr34 is the key to maintaining the convoluted structure of the CC′ loop as shown for msCEACAM1a[1,4]. Thus, the CC′ loops of CEA and human CEACAM1 probably assume a convoluted conformation like that of msCEACAM1a[1,4]. The second point is that the area around Phe29 of CEA and Ile91 of human CEACAM1 (corresponding to Gly29 and Thr91 in msCEACAM1a[1,4], FIG. 2B) is highly hydrophobic and might be an important determinant of binding energy. Knowing the structure of msCEACAM1a[1,4] makes it possible to rationally design mutations to elucidate the molecular basis of the specific interactions between bacterial Opa proteins and CEA members on human cell membranes. Based on the CEACAM1 structure, it is possible to design small molecules that can interfere with binding of ligands to the biologically important CC′ loop of CEACAM1 or related CEA family members.

### EXAMPLE 9

Pregnancy-Regulating Drug Selection

The pregnancy-specific glycoprotein (PSG) subfamily of the CEA family appears to be essential for a successful pregnancy, although the functions of PSGs are not yet fully understood. PSGs may attenuate the mother's immune response to her semi-allogeneic fetus (Hammarstrom, 1999). The N-terminal domains of most human PSGs, but not baboon or rodent PSGs, contain an Arg-Gly-Asp (RGD) motif. The RGD motif is known to be associated with integrin binding and mediates a wide variety of cell adhesion events. For example, in human fibronectin (FN), an integrin-binding RGD motif is located on a type II′ turn at the tip of a protruded FG loop of the 10^{th} FN domain (Leahy et al., 1996). FIG. 4A shows that in D1 of the human PSGs the RGD motifs are aligned at the very tip of the FG loop (highlighted in violet in FIG. 1). The corresponding sequence in msCEACAM1a[1,4] is Glu92-Asn93-Tyr94 (FIG. 4A), which assumes a type II β turn. Those PSG proteins with an RGD motif can slightly change the conformation at the tip of the FG loop to adopt a type II′ turn more suitable for integrin binding. The heterophilic binding of soluble PSGs to integrins might cause local immunosuppression in the uterus by shielding the integrins on cell membranes (Hammarstrom, 1999). In other species, PSGs lacking the RGD motif may still use one acidic residue (Glu or Asp) in the protruding FG loop (Zhou and Hammarstrom, 2001) to bind integrin, as demonstrated for leukocyte integrin ligands (Wang and Springer, 1998) and E-cadherin (Taraszka et al., 2000).

CEA family members can mediate intercellular adhesion in vitro and in vivo through binding interactions that involve the N-terminal domain (Hammarstrom, 1999). Mutational analyses of the N-terminal domain (D1) of human CEACAM1 and CEA showed that residues on the CFG face, and especially residues on the CC′ loop of D1 are directly engaged in homophilic cell adhesion. Mutations V39A and D40A in the CC′ loop abolished homophilic adhesion of human CEACAM1.

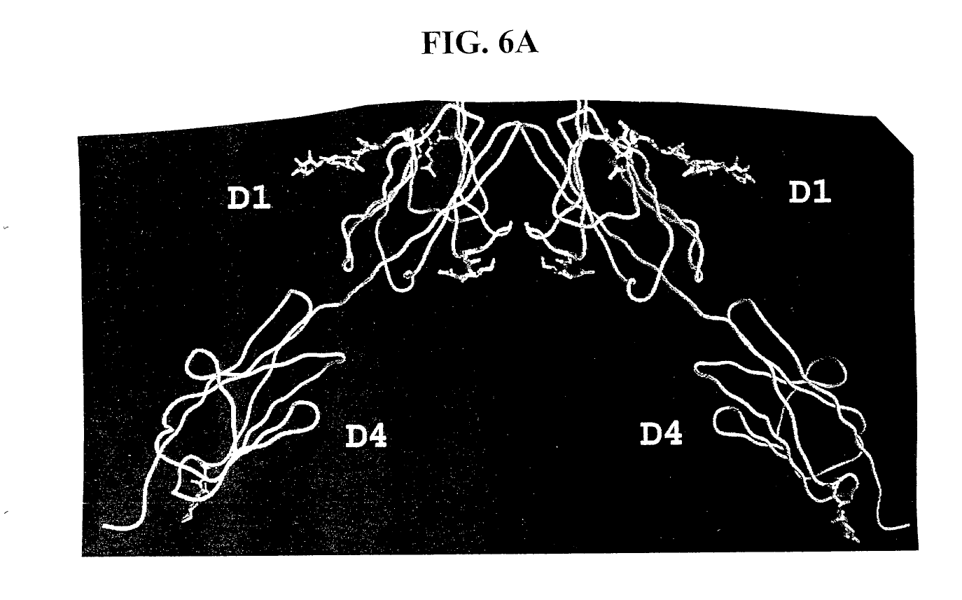

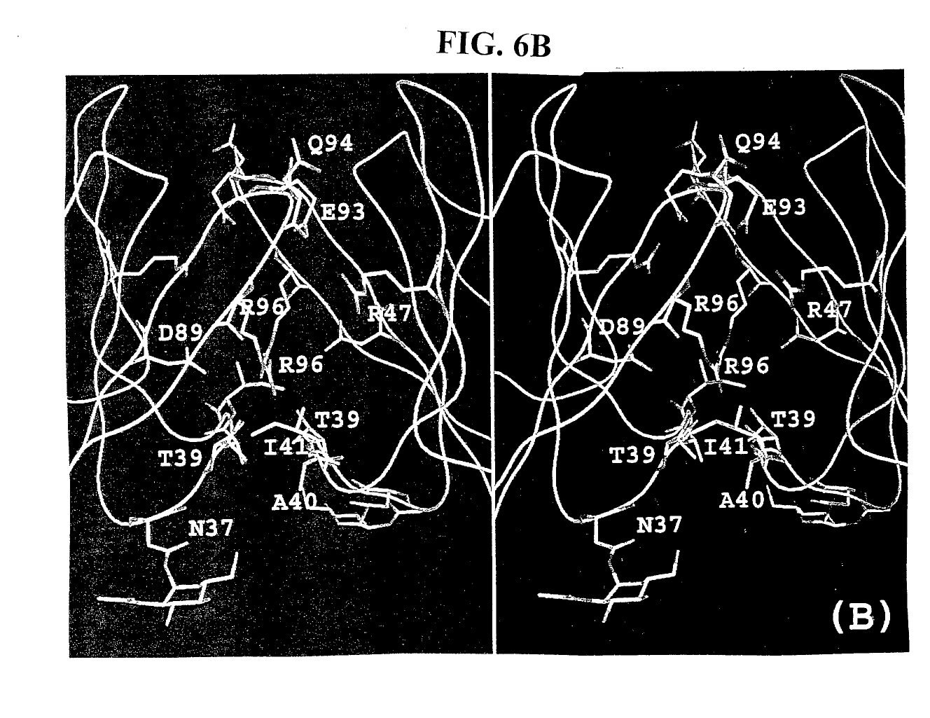

To study mechanisms for homophilic binding of msCEACAM1a[1,4], the molecular interactions observed in the crystal lattice of msCEACAM1a[1,4] were examined. Two major contact areas between symmetry-related molecules were found, one through D1 by a 2-fold axis, and the other through D4 by a 3-fold axis. The D1-D1 contact seems most interesting. FIG. 6 shows how the CC′ and FG loops in D1s of two dyad-related molecules made contact in the crystal structure of msCEACAM1a[1,4]. Hydrophilic interactions appear to dominate the adhesive interface, like that between CD2 and CD58 (Wang et al., 1999). However, the D1-D1 contact seen in FIG. 6 is quite different from the anti-parallel “hand-shaking” mode of CD2/CD58 interactions via their relatively flat CFG faces. For several reasons, the more “parallel” mode of homophilic D1-D1 contact seen between msCEACAM1a proteins are considered by the present inventors to be of physiological significance. First, as discussed above, the uniquely convoluted conformation of the CC′ loop of msCEACAM1a[1,4] is likely to be similar for human CEA members. The fact that Y34A, but not Y34F, mutation abrogated homophilic adhesion of CEA (Taheri et al., 2000) shows the importance of the hydrophobic aromatic ring for maintaining the structure of the convoluted CC′ loop. A convoluted, protruding CC′ loop would likely prevent CEA molecules from adopting the “hand-shaking” type of adhesion seen between CD2 and CD58. FIG. 6B shows that Val39 of one human CEACAM1 molecule (corresponding to Thr39 in msCEACAM1a[1,4]) might have hydrophobic contact with Val39 from its symmetry-mate, while Asp40 of CEA (corresponding to Ala40 of msCEACAM1a[1,4], FIG. 6B) might potentially form a salt bridge with Arg38 from the symmetry-mate. This may explain why mutations V39A and D40A in CEACAM1 disrupt homophilic cell adhesion.

The “parallel” mode of adhesion could occur between molecules on the same cell or opposing cells. The numerous inter-domain junctions of long CEA members may render them flexible enough to permit a trans-interaction between opposing cells using this “parallel” mode. An example is membrane fusion of eukaryotic cells that is mediated by the trans-SNARE complex. R-SNARE and Q-SNARE components from opposing cells come together to form a helical bundle in a “parallel” mode (Mayer, 2001). CHO cells transfected with human CEACAM1-1s, which has only the D1 domain as its extra-cellular portion, showed negligible adhesion despite a high level of protein. Not enough flexibility in this short molecule prohibited this “parallel” mode of binding. Further crystallographic studies and mutational analysis are needed to characterize cis- or trans-adhesion mechanisms between CEA family members.

### EXAMPLE 10

Drug Screening for Anti-Viral, Anti-Inflammatory and Anti-Cancer Agents

The present example is provided to demonstrate the utility of the present invention for the selection and screening a variety of candidate substances for anti-viral, anti-inflammatory, and anti-cancer activity.

The target control molecule that will be used is the soluble carcnoembryonic antigen (CEA) described herein. The agent that will be used to quantify binding activity of a candidate substance, and against which the relative acceptability of a candidate substance will be determined, will be a monoclonal antibody, CC1. One such monoclonal antibody is described in Wessner at al. (1998)(J. Virol, 72(3):1941-48)), which reference is specifically incorporated herein by reference. In general, substances (i.e., a candidate substance) that is capable of binding specifically to the CC′ loop of CEACAM1 having the uniquely conformational characteristics identified here with an binding affinity in the range of 10(4) to 10(10) will be selected for use as a potentially suitable anti-viral, anti-inflammatory and/or anti-cancer agent.

It should be understood that other monoclonal and polyclonal antibodies, or other types of molecules, that posses the same or relatively the same binding affinity for the novel structure of the CC′ loop of mouse or human CEACAM1 protein as described here may also be used in the practice of the method for selecting candidate substances suitable for the uses described here.

It is expected that the disclosed method will be useful in identifying agents that may be used in the treatment and therapy of humans using the identified functional domain of CEACAM1 identified here as the CC′ loop because of the high degree of structural similarity that the present investigators have inferred from mutational data as existing between the sequenced CC′ region of mouse and human CEACAM1a. This region possesses about 10 amino acids in the mouse and the human sequences which are compared below, along with the amino acids that stabilize the uniquely structure of the CC′ loop:

MouseCC′ region——KGNTTAIDKE-

Important amino acids that stabilize the structure of the CC′ loop:

Y34, E44, R47, R96 and possibly D89

Human CC′ region——K G E R V D G N R Q—(SEQ ID NO: 1)

Amino acids that likely stabilize the structure of the CC′ loop:

Y34, Q44, G47, and Q89

It is envisioned that the structure of this loop will be reduced to an algorithm that will provide a three-dimensional (3-D) blueprint of structure against which candidate substances can be compared and identified as likely to attach to the D1 functional domain, CC′. This will then be incorporated into a software program wherein the calculation and identification of likely suitable candidate substances can be screened automatically and at a relatively rapid rate. Software programs currently available in the art for the purpose of drug screening and selection may be found at http://www.small-molecule-drug-discovery.com/high screening.html.

The identified candidate substances that have the activity for binding as identified here, are also intended as part of the present invention. As a further step, and in some embodiments, the selected candidate substances may then be examined in an in vitro assay, such as for ability to bind CEACAM1 protein. Specificity of binding will be tested by using CEACAM1 proteins from different species, and other related glycoproteins in the CEA family.

Alternatively, the candidate substance can be tested for the ability to block the binding of a monoclonal antibody such as anti-CEACAM1 Mab-CC1 or the MHV viral spike glycoprotein (S) or a homophilic region of CEACAM1 to the functional domain CC′ of the CEACAM1 protein.

In yet another approach, the candidate substance may be tested for its ability to block the binding of MHV to mCEACAM1a, or for the ability to block the homophilic interaction of mCEACAM1a.

TABLE 1
Data Collection, Structure determination and Refinement
Data Collection

| Data set | Pt peak^{¶} | Pt-inflection^{¶} | Pt-remote | Native |
|---|---|---|---|---|
| Space group | | | P3₁21 | |
| Unit Cell (Å) | | a, b = 111.85, | | a, b = 111.3, |
| | | c = 66.34 | | c = 65.67 |
| X-ray source | | | APS | |
| Wavelength (Å) | 1.0715 | 1.0718 | 1.0534 | 1.100 |
| Resolution (Å) | 20-3.85 | 20-3.85 | 20-3.85 | 20-3.3 |
| Observations | 49179 | 50389 | 45774 | 123640 |
| (uniquely) | (8681)^{¶} | (8645)^{¶} | (8566) | (7127) |
| I/σ overall | 16.0 | 15.2 | 13.2 | 17.3 |
| | (3.1)* | (3.3)* | (2.3)* | (3.7)* |
| Completeness (%) | 99.2 | 99.6 | 97.6 | 99.7 |
| | (91.8)* | (96.3)* | (82.9)* | (100.0)* |
| R_{Merge} (%) | 7.5 | 6.9 | 8.0 | 7.3 |
| | (45.4)* | (42.3)* | (55.4)* | (37.1)* |

Structure Determination

| | | | |
|---|---|---|---|
| Figure of Merit | | 0.49 | |
| Phasing power | 1.92 | 1.86 | 1.79 |
| R_{Cullis} | 0.82 | 0.84 | 0.88 |
| (anomalous) | | | |
| R_{Cullis} | 0.60 | 0.61 | 0.61 |
| (isomorpous) | | | |

Structure Refinement

| | |
|---|---|
| Resolution (Å) | 15-3.3 |
| Number of work/test reflections | 6144/754 |
| Nonhydrogen protein/carbohydrate/solvent atoms | 1692/81/26 |
| R_{Work}/R_{Free} (%) | 29.5/32.9 |
| Bond length (Å)/angle (°) rms deviation from ideal | 0.011/2.325 |
| geometry | |
| Ramachandran statistics (%) | 68.5/23.4/8.2/0 |
| Favourable/Additional/Generous/Forbidden | |
| Protein atoms average B value (Å²), Mainchain/ | 55.12/64.15 |
| Sidechain | |

^{¶}Bijvoet pairs are both counted
(Last resolution bin)

### EXAMPLE 11

Pharmaceutical Preparations for Angiogenesis and Tumor Inhibition

The molecules of the present invention may be selected to provide a pharmacologically active preparations that will provide interference with aberrant angiogenesis, tumor metastasis inhibition, or other functions. Because MAb-CC1 in the circulation inhibits delayed type hypersensitivity in vivo (and blocks MHV virus binding to CEACAM1 on murine cells) and virus binds by the CC′ loop, the CC′ loop is an important biological molecule needed for delayed type hypersensitivity in vivo. Inhibiting/blocking this loop on D1 may prevent DTH or other immune mediated damage. This could be used in allergic reactions, autoimmune disorders etc. The other application for pharmacological uses focuses on the angiogenesis activity of CEACAM1.

Bibliography

The following bibliography articles are specifically incorporated herein by reference:

Bates, P. A., Luo, J., and Sternberg, M. J. (1992). A predicted three-dimensional structure for the carcinoembryonic antigen (CEA), FEBS Lett 301, 207-14.

Beauchemin, N., Draber, P., Dveksler, G., Gold, P., Gray-Owen, S., Grunert, F., Hammarstrom, S., Holmes, K. V., Karlsson, A., Kuroki, M., et al. (1999). Redefined nomenclature for members of the carcinoembryonic antigen family, Exp Cell Res 252, 243-249.

Bergelson, J. M., Cunningham, J. A., Droguett, G., Kurt-Jones, E. A., Krithivas, A., Hong, J. S., Horwitz, M. S., Crowell, R. L., and Finberg, R. W. (1997). Isolation of a common receptor for Coxsackie B viruses and adenoviruses 2 and 5, Science 275, 1320-3.

Bos, M. P., Hogan, D., and Belland, R. J. (1999). Homologue scanning mutagenesis reveals CD66 receptor residues required for neisserial Opa protein binding, J Exp Med 190, 331-40.

Brunger, A. T. (1992). X-PLOR. Version 3.1: a system for crystallography and NMR. (New Haven, Yale University press,).

Casasnovas, J. M., Larvie, M., and Stehle, T. (1999). Crystal structure of two CD46 domains reveals an extended measles virus-binding surface [In Process Citation], Embo J 18, 2911-22.

Casasnovas, J. M., Springer, T. A., Liu, J. H., Harrison, S. C., and Wang, J. -H. (1997). Crystal structure of ICAM-2 reveals a distinctive integrin recognition surface, Nature 387, 312-5.

CCP4 (1994). The CCP4 suite: programs for protein crystallography, Acta Crystallogr D50, 760-763.

Chothia, C., Gelfand, I., and Kister, A. (1998). Structural determinants in the sequences of immunoglobulin variable domain, J Mol Biol 278, 457-79.

Clackson, T., and Wells, J. A. (1995). A hot spot of binding energy in a hormone-receptor interface, Science 267, 383-6.

Dveksler, G. S., Dieffenbach, C. W., Cardellichio, C. B., McCuaig, K., Pensiero, M. N., Jiang, G. S., Beauchemin, N., and Holmes, K. V. (1993a). Several members of the mouse carcinoembryonic antigen-related glycoprotein family are functional receptors for the coronavirus mouse hepatitis virus-A59, J Virol 67, 1-8.

Dveksler, G. S., Pensiero, M. N., Cardellichio, C. B., Williams, R. K., Jiang, G. S., Holmes, K. V., and Dieffenbach, C. W. (1991). Cloning of the mouse hepatitis virus (MHV) receptor: expression in human and hamster cell lines confers susceptibility to MHV, J Virol 65, 6881-91.

Dveksler, G. S., Pensiero, M. N., Dieffenbach, C. W., Cardellichio, C. B., Basile, A. A., Elia, P. E., and Holmes, K. V. (1993b). Mouse hepatitis virus strain A59 and blocking antireceptor monoclonal antibody bind to the N-terminal domain of cellular receptor, Proc Natl Acad Sci U S A 90, 1716-20.

Epp, O., Lattman, E. E., Schiffer, M., Huber, R., and Palm, W. (1975). The molecular structure of a dimer composed of the variable portions of the Bence-Jones protein REI refined at 2.0-A resolution, Biochemistry 14, 4943-52.

Ergun, S., Kilik, N., Ziegeler, G., Hansen, A., Nollau, P., Gotze, J., Wurmbach, J. H., Horst, A., Weil, J., Fernando, M., and Wagener, C. (2000). CEA-related cell adhesion molecule 1: a potent angiogenic factor and a major effector of vascular endothelial growth factor, Mol Cell 5, 311-20.

Gallagher, T. M. (1997). A role for naturally occurring variation of the murine coronavirus spike protein in stabilizing association with the cellular receptor, J Virol 71, 3129-37.

Gold, P., and Freedman, S. O. (1965). Specific carcinoembryonic antigens of the human digestive system, J Exp Med 122, 467-81.

Hammarstrom, S. (1999). The carcinoembryonic antigen (CEA) family: structure, suggested functions and expression in normal and malignant tissues, Vol 9, Academic Press), pp. 67-81.

Harpaz, Y., and Chothia, C. (1994). Many of the immunoglobulin superfamily domains in cell adhesion molecules and surface receptors belong to a new structural set which is close to that containing variable domains, J Mol Biol 238, 528-39.

He, T. C., Zhou, S., da Costa, L. T., Yu, J., Kinzler, K. W., and Vogelstein, B. (1998). A simplified system for generating recombinant adenoviruses, Proc Natl Acad Sci U S A 95, 2509-14.

Hemmingsen, J. M., Gernert, K. M., Richardson, J. S., and Richardson, D. C. (1994). The tyrosine corner: a feature of most Greek key beta-barrel proteins, Protein Sci 3, 1927-37.

Huang, J., Hardy, J. D., Sun, Y., and Shively, J. E. (1999). Essential role of biliary glycoprotein (CD66a) in morphogenesis of the human mammary epithelial cell line MCF10F, J Cell Sci 112, 4193-205.

Huber, M., Izzi, L., Grondin, P., Houde, C., Kunath, T., Veillette, A., and Beauchemin, N. (1999). The carboxyl-terminal region of biliary glycoprotein controls its tyrosine phosphorylation and association with protein-tyrosine phosphatases SHP-1 and SHP-2 in epithelial cells, J Biol Chem 274, 335-44.

Izzi, L., Turbide, C., Houde, C., Kunath, T., and Beauchernin, N. (1999). cis-Determinants in the cytoplasmic domain of CEACAM1 responsible for its tumor inhibitory function, Oncogene 18, 5563-72.

Jones, E. Y., Davis, S. J., Williams, A. F., Harlos, K., and Stuart, D. I. (1992). Crystal structure at 2.8 A resolution of a soluble form of the cell adhesion molecule CD2, Nature 360, 232-9.

Jones, T. A., Zou, J. -Y., Cowan, S. W., and Kjeldgaard, M. (1991). Improved methods for building protein models in electron density maps and location of errors in these models, Acta Crystallogr A47, 110-119.

Kolatkar, P. R., Bella, J., Olson, N. H., Bator, C. M., Baker, T. S., and Rossmann, M. G. (1999). Structural studies of two rhinovirus serotypes complexed with fragments of their cellular receptor, Embo J 18, 6249-59.

Krulis, P. (1991). MOLSCRIPT: a program to produce both detailed and schematic plots, J Appl Cryst 24, 924-950.

Kwong, P. D., Wyatt, R., Robinson, J., Sweet, R. W., Sodroski, J., and Hendrickson, W. A. (1998). Structure of an HJV gp120 envelope glycoprotein in complex with the CD4 receptor and a neutralizing human antibody [see comments], Nature 393, 648-59.

Leahy, D. J., Aukhil, I., and Erickson, H. P. (1996). 2.0 Å crystal structure of a four-domain segment of human fibronectin encompassing the RGD loop and synergy region, Cell 84, 155-164.

Mayer, A. (2001). What drives membrane fusion in eukaryotes?, Trends Biochem Sci 26, 717-723.

Morales, V. M., Christ, A., Watt, S. M., Kim, H. S., Johnson, K. W., Utku, N., Texieira, A. M., Mizoguchi, A., Mizoguchi, E., Russell, G. J., et al. (1999). Regulation of human intestinal intraepithelial lymphocyte cytolytic function by biliary glycoprotein (CD66a), J Immunol 163, 1363-70.

Nedellec, P., Dveksler, G. S., Daniels, E., Turbide, C., Chow, B., Basile, A. A., Holmes, K. V., and Beauchemin, N. (1994). Bgp2, a new member of the carcinoembryonic antigen-related gene family, encodes an alternative receptor for mouse hepatitis viruses, J Virol 68, 4525-37.

Nicholls, A., Sharp, K. A., and Honig, B. (1991). Protein folding and association: insights from the interfacial and thermodynamic properties of hydrocarbons, Proteins 11, 281-96.

Ohtsuka, N., Yamada, Y. K., and Taguchi, F. (1996). Difference in virus-binding activity of two distinct receptor proteins for mouse hepatitis virus, J. Gen Virol 77, 1683-92.

Otwinowski, Z., and Minor, W. (1997). Processing of X-ray diffraction data collected in oscillation mode. In Macromolecular Crystallography, C. W. Carte Jr., and R. M. Sweet, eds. (San Diego, London, Boston, N.Y., Syney, Tokyo, Toronto, Academic Press), pp. 307-326.

Rao, P. V., Kumari, S., and Gallagher, T. M. (1997). Identification of a contiguous 6-residue determinant in the MHV receptor that controls the level of virion binding to cells, Virology 229, 336-48.

Remington's Pharmalogical Basis of Therapeutices (1997).

Sambrook, J. Russel D., Molecular Cloning: A Laboratory Manual Third Edition. Cold Spring Harbor Laboratory Press. Cold Spring Harbor, N.Y., 2001

Stanley, P. (1989). Chinese hamster ovary cell mutants with multiple glycosylation defects for production of glycoproteins with minimal carbohydrate heterogeneity, Mol Cell Biol 9, 377-83.

Stewart, J. J., Lee, C. Y., Ibrahim, S., Watts, P., Shlomchik, M., Weigert, M., and Litwin, S. (1997). A Shannon entropy analysis of immunoglobulin and T cell receptor, Mol Immunol 34, 1067-82.

Stuart, D. I., and Jones, E. Y. (1995). Recognition at the cell surface: recent structural insights, Curr Opin Struct Biol 5, 735-43.

Taheri, M., Saragovi, U., Fuks, A., Makkerh, J., Mort, J., and Stanners, C. P. (2000). Self recognition in the Ig superfamily. Identification of precise subdomains in carcinoembryonic antigen required for intercellular adhesion, J Biol Chem 275, 26935-43.

Talbot, P. J., and Buchmeier, M. J. (1985). Antigenic variation among murine coronaviruses: evidence for polymorphism on the peplomer glycoprotein, E2, Virus Res 2, 317-28.

Taraszka, K. S., Higgins, J. M., Tan, K., Mandelbrot, D. A., Wang, J. H., and Brenner, M. B. (2000). Molecular basis for leukocyte integrin alpha(E)beta(7) adhesion to epithelial (E)-cadherin, J Exp Med 191, 1555-67.

Virji, M., Evans, D., Griffith, J., Hill, D., Serino, L., Hadfield, A., and Watt, S. M. (2000). Carcinoembryonic antigens are targeted by diverse strains of typable and non-typable Haemophilus influenzae, Mol Microbiol 36, 784-95.

Virji, M., Evans, D., Hadfield, A., Grunert, F., Teixeira, A. M., and Watt, S. M. (1999). Critical determinants of host receptor targeting by Neisseria meningitidis and Neisseria gonorrhoeae: identification of Opa adhesiotopes on the N-domain of CD66 molecules, Mol Microbiol 34, 538-51.

Wang, J. -H., Smolyar, A., Tan, K., Liu, J. -H., Kim, M., Sun, Z. -Y. J., Wagner, G., and E. L., R. (1999). Structure of a heterophilic adhesion complex between human CD2 and CD58 (LFA-3) counter-receptors, Cell 97, 791-803.

Wang, J. -H., and Springer, T. A. (1998). Structural specializations of immunoglobulin superfamily members for adhesion to integrins and viruses, Immunological Review 163, 197-215.

Wang, J. -H., Yan, Y. W., Garrett, T. P., Liu, J. H., Rodgers, D. W., Garlick, R. L., Tarr, G. E., Husain, Y., Reinherz, E. L., and Harrison, S. C. (1990). Atomic structure of a fragment of human CD4 containing two immunoglobulin-like domains [see comments], Nature 348, 411-8.

Wessner, D. R., Shick, P. C., Lu, J. H., Cardellichio, C. B., Gagneten, S. E., Beauchemin, N., Holmes, K. V., and Dveksler, G. S. (1998). Mutational analysis of the virus and monoclonal antibody binding sites in MHVR, the cellular receptor of the murine coronavirus mouse hepatitis virus strain A59, J Virol 72, 1941-8.

Wu, H., Kwong, P. D., and Hendrickson, W. A. (1997). Dimeric association and segmental variability in the structure of human CD4, Nature 387, 527-30.

Zelus, B. D., Wessner, D. R., Williams, R. K., Pensiero, M. N., Phibbs, F. T., deSouza, M., Dveksler, G. S., and Holmes, K. V. (1998). Purified, soluble recombinant mouse hepatitis virus receptor, Bgp1(b), and Bgp2 murine coronavirus receptors differ in mouse hepatitis virus binding and neutralizing activities, J Virol 72, 7237-44.

What is claimed is:

1. A method for screening a candidate substance for binding to and/or inhibiting binding to CEACAM1 or a structurally related CEA family member of a ligand or inhibiting a biological activity such as cell adhesion, tumor metastasis, angiogenesis, virus binding and infection, or (bacterial inhibiting, or cell adhesion inhibiting) activity comprising:
preparing a soluble CEACAM1 protein comprising a functional binding domain, D1, having a protruding, convoluted CC′ loop amino acid sequence for humans of K G E R V D G N R Q); a C-TERMINAL domain, D4, having an elongated CD loop, and a flexible linker connecting D1 to D4, to provide a target protein;
preparing a control sample comprising the target protein and a monoclonal antibody having specific binding affinity for the CC′ loop, and preparing a test sample comprising the target protein and a candidate substance;
incubating the control sample and the test sample for a period of time and under appropriate conditions to permit binding to the target protein in the control sample; and
comparing the amount of antibody-bound target protein in the control sample to the amount of candidate agent bound target protein in the test sample, wherein a candidate agent having at least 40% the amount of bound candidate agent to target protein compared to the amount of bound target protein in the control sample is selected as having sufficient binding/inhibiting activity.

2. The method of claim 1 wherein D1 further comprises a first and a second anti-parallel beta-sheet connected to one another by a salt bridge.

3. The method of claim 1 wherein the ligand is a homophilic binding domain of CEACAM1, MHV viral spike glycoprotein, Neisseria, or Hemophilus bacteria.

4. The method of claim 1 wherein the target protein comprises a cell surface receptor.

5. The method of claim 4 wherein the target protein comprises a cell surface protein on an epithelial cell, a leukocyte, an endothelial cell, or a placental cell.

6. The method of claim 1 wherein the selected candidate substance inhibits virus binding.

7. The method of claim 3 wherein the selected candidate substance inhibits binding of a pathogenic strain of bacteria of Neisseria or Hemophilus.

8. The method of claim 7 wherein the pathogenic strain is a Hemophilus strain.

9. The method of claim 7 wherein the pathogenic strain of bacteria is a Hemophilus strain.

10. The method of claim 1 wherein the selected candidate substance is capable of blocking cell-mediated immune responses.

11. The method of claim 1 wherein the selected candidate substance provides a bacterial inhibiting activity.

12. The method of claim 10 wherein the selected candidate substance provides a treatment for bacterial infection.

13. The method of claim 10 wherein the selected candidate substance provides a treatment for diarrhea.

14. The method of claim 10 wherein the selected candidate substance provides a treatment for hepatitis.

15. A soluble protein in the CEA family comprising:
a hydrophobic core molecule;
a functional CC′ binding domain having a convoluted and protruding structure; and
a carboxy terminal D4 containing an elongated CD loop.

16. The soluble CEA family protein of claiml4 further defined as having an A-A′ kink comprising a cis-proline amino acid residue.

17. The soluble CEA family protein of claim 14 further comprising a detectable molecular tag molecule.

18. The soluble CEA family protein of claim 14 further defined as comprising an amino acid sequence of SEQ ID NO: 1.

19. The soluble CEA family protein of claim 14 further defined as comprising an amino acid sequence of SEQ ID NO: 2.

20. The soluble CEA family protein of claim 14 further defined as comprising an amino acid sequence of SEQ ID NO: 3.

21. The soluble CEA family protein of claim 15 further defined as a cellular receptor for a coronavirus.

22. A pharmaceutical formulation comprising the molecule of claim 15 in a pharmaceutically acceptable excipient.

23. The pharmaceutical formulation of claim 22 further defined as an antiviral agent.

24. An antiviral agent comprising a molecule capable of binding with high affinity and under stringent conditions to a target antigen molecule having:
a virus binding domain, D1, having a first and a second anti-parallel beta-sheet connected to one another by a salt bridge, a protruding, convoluted CC′ loop, and an A-A′ kink,
a C-terminal domain, D4, having an elongated CD loop, and
a flexible linker connecting D1 to D4.

25. The antiviral agent of claim 24 wherein the anti-viral agent is further defined as binding to the target antigen molecule with an affinity of about 10(4) to about 10(10).
